(19) United States
(12) Patent Application Publication
(10) Pub. No.: US 2009/0180951 A1
(43) Pub. Date: Jul. 16, 2009

(54) INHIBITORS OF INTEGRIN VLA-4

(75) Inventors: Craig N. ZIMMERMAN, Topsfield, MA (US); John W. BABICH, Cambridge, MA (US)

Correspondence Address: FOLEY AND LARDNER LLP;SUITE 500, 3000 K STREET NW, WASHINGTON, DC, 20007, US

(73) Assignee: Molecular Insight Pharmaceuticals, Inc.

(21) Appl. No.: 12/331,747

(22) Filed: Dec. 10, 2008

Related U.S. Application Data
(60) Provisional application No. 60/996,963, filed on Dec. 12, 2007.

(51) Int. Cl. A61K 51/00; C07F 13/00; A61P 29/00

(52) U.S. Cl. 424/1.65; 534/14

(57) ABSTRACT

A complex, its stereoisomer or pharmaceutically acceptable salt has the formula I, where:

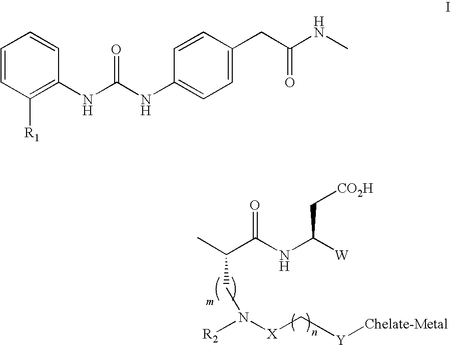

wherein:
   W is a group that is A, B, or C:

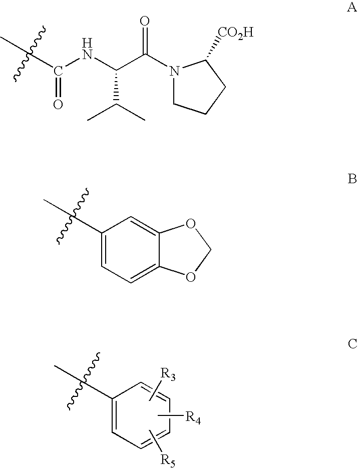

R₁ is hydrogen, alkyl or alkoxy;
R₂ is a bond, hydrogen or alkyl
R₃, R₄ and R₅ are independently hydrogen, iodine, alkyl, alkoxy, hydroxyl, amino, aminoalkyl, dialkylamino, or carboxyl;
X is a bond, C═O, O═C—O, or CH₂;
Y is a bond, CH₂, or O;
m is an integer ranging from 1 to 6;
n is an integer ranging from 0 to 6;
Metal represents a metallic moiety comprising a radionuclide; and
Chelate represents a chelating moiety that coordinates with said radionuclide to form the complex.

### CROSS-REFERENCE TO RELATED APPLICATIONS

This application claims the benefit of U.S. Provisional Patent Application No. 60/996,963, filed Dec. 12, 2007, the entire contents of which are incorporated herein by reference for any and all purposes.

### FIELD

This invention generally relates to radiopharmaceuticals for diagnostic imaging and therapeutic treatment of diseases, and in particular, to radiolabeled inhibitors of integrin VLA-4 for the treatment and diagnosis of VLA-4 expressing diseases.

### BACKGROUND

Integrins are cell surface receptors that interact with the extracellular matrix and mediate various intracellular signals. Integrins are non-covalent heterodimeric complexes consisting of two subunits called α and β. There are at least 12 different α subunits (α1-α6, α-L, α-M, α-X, α-IIB, α-V and α-E) and at least 9 different β (β1-β9) subunits. Based on the type of its α and β subunit components, each integrin molecule is categorized into a subfamily. Integrin α4β1, also known as very late antigen-4 (VLA-4), is a leukocyte cell surface receptor that participates in a wide variety of cell-cell and cell-matrix adhesive interactions. It mediates leukocyte recruitment, activation, mediator release, and apoptosis inhibition.

VLA-4 plays a central role in inflammatory pathophysiology and is an emerging target for the treatment of a variety of inflammatory processes. VLA-4 also plays critical roles in angiogenesis in a variety of cancers. Of particular interest is the VLA-4's role in acute myelogenous leukemia (AML). VLA-4 has been shown to be the primary cause of minimal residual disease in relapse AML after chemotherapy. Its role in AML as well as in angiogenesis in general makes it an ideal target for radioimaging and radiotherapy.

### SUMMARY

This invention provides novel radiopharmaceuticals that are useful in diagnostic imaging and therapeutic treatment of disease which is characterized by expressing VLA-4. The radiopharmaceuticals comprise a complex that contains a molecule moiety which is capable of inhibiting or suppressing VLA-4 adhesive activity, and a radionuclide adapted for radioimaging and/or radiotherapy.

In one aspect, a complex of formula I, its stereoisomer or pharmaceutically acceptable salt is provided:

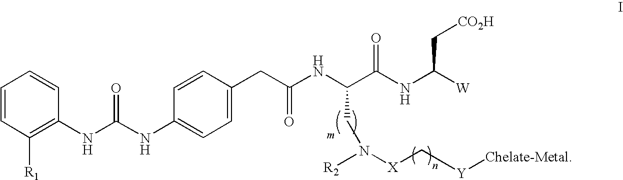

In formula I, W is A, B, or C:

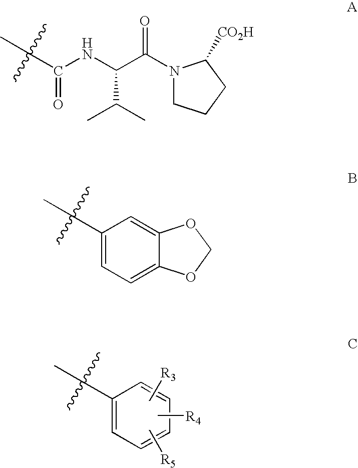

where R₁ is hydrogen, alkyl or alkoxy;
R₂ is a bond, hydrogen or alkyl;
R₃, R₄ and R₅ are independently hydrogen, iodine, alkyl, alkoxy, hydroxyl, amino, aminoalkyl, dialkylamino, or carboxyl;
X is a bond, C═O, O═C—O, or CH₂;
Y is a bond, CH₂, or O;
m is an integer ranging from 1 to 6;
n is an integer ranging from 0 to 6;
Metal represents a metallic moiety comprising a radionuclide; and
Chelate represents a chelating moiety that coordinates with said radionuclide to form the complex.

In some embodiments, the radionuclide is selected from the group consisting of technetium, rhenium, yttrium, indium, gallium, gadolinium, and copper.

In some embodiments, the Chelate is selected from the group consisting of 1,4,7,10-tetraazacyclododecane-1,4,7,10-tetraacetic acid (DOTA), diethylenetriaminepentaacetic acid (DTPA), pyridylmethylene amine (PDA), quinolinemethylene amine, isoquinoline amine, pyridine-2-ylmethylamino acetic acid (PAMA), isoquinolin-3-ylmethylamino acetic acid, thiazol-2-ylmethyl amine, thiazol-2-ylmethylamino acetic acid, N-methylimidazole(methylene)amine, N-methylimidazole(methylene)amino acetic acid, NOTA, Hynic, MAG3, N₂S₂, MAMA and DADT.

In some embodiments, the Metal-Chelate moiety is D, E, F, G, H, or J:

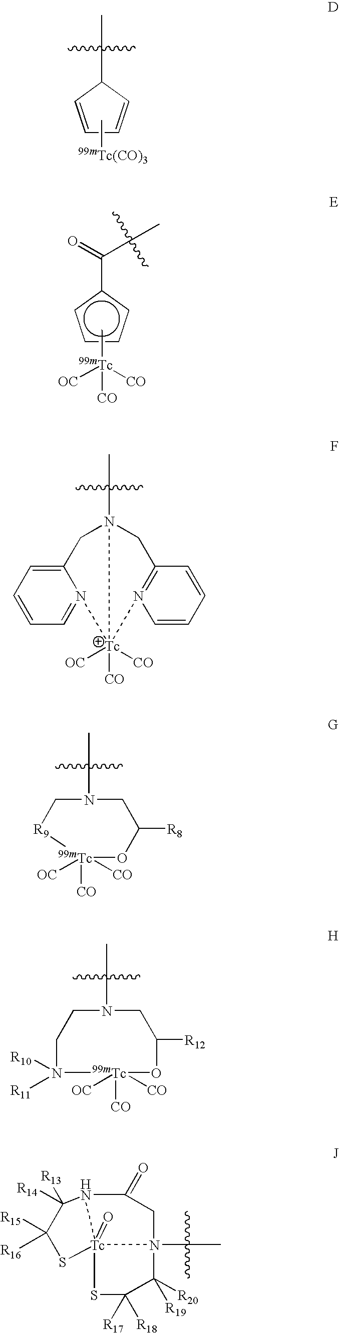

where R₈ is selected from the group consisting of O, H, OH, alkoxy, or O-alkyl;
R₉ is a pharmaceutically acceptable 5 or 6-member heterocyclic ring;
R₁₀ and R₁₁ are each independently hydrogen, alkyl, or substituted alkyl;
R₁₂ is selected from the group consisting of aryl, alkyl, or heterocycle; and
R₁₃, R₁₄, R₁₅, R₁₆, R₁₇, R₁₈, R₁₉, R₂₀ are independently hydrogen or methyl.

In some embodiments, the complex of formula I has the structure is that of I-a, I-b, I-c, I-d, I-e, or I-f:

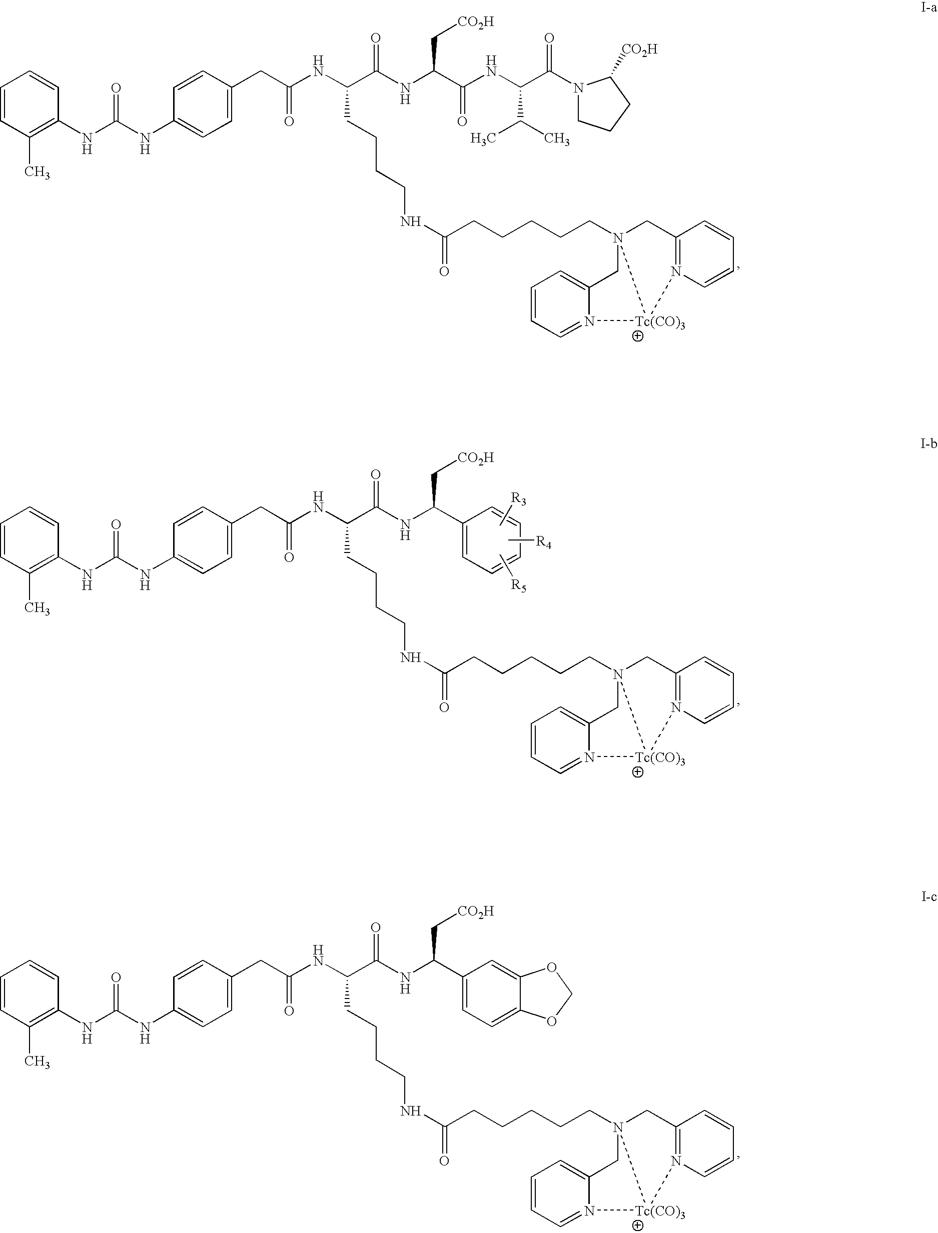

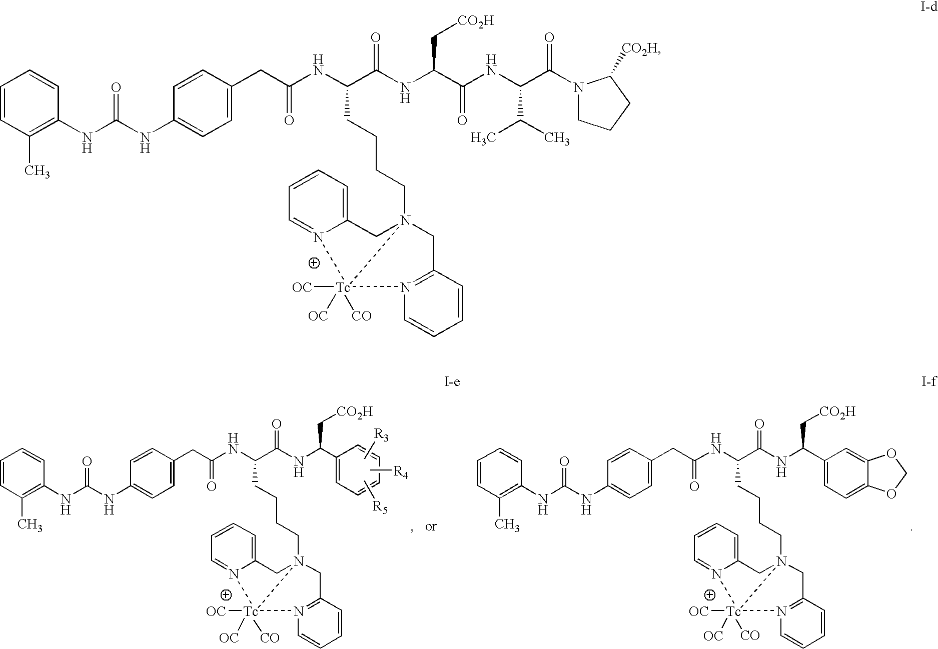

In formulas I-a to I-f, R₃, R₄ and R₅ are independently hydrogen, iodine, alkyl, alkoxy, hydroxyl, amino, aminoalkyl, dialkylamino, or carboxyl.

In another aspect, the invention provides a method of imaging tissue. In one embodiment, the tissue expresses VLA-4. The method comprises administering to the mammal an effective amount of a complex represented by formula I and attendant definitions.

In a further aspect, the invention provides a method of treating a mammal suffering a disease which is characterized by expressing VLA-4. The method comprises administering to the mammal a therapeutically effective amount of a complex represented by formulas I and attendant definitions.

In still another aspect, a kit is provided comprising the subject complexes and a pharmaceutically acceptable carrier, and optionally instructions for their use. Uses for such kids include therapeutic management and medical imaging applications.

### DETAILED DESCRIPTION

Various embodiments of the invention are described hereinafter. It should be noted that the specific embodiments are not intended as an exhaustive description of the invention or as a limitation on the scope of the invention. One aspect described in conjunction with a particular embodiment of the present invention is not necessarily limited to that embodiment and can be practiced with any other embodiment(s) of the invention.

As used herein, the following definitions of terms shall apply unless otherwise indicated.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The embodiments, illustratively described herein may suitably be practiced in the absence of any element or elements, limitation or limitations, not specifically disclosed herein. Thus, for example, the terms “including,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed invention. Additionally the phrase “consisting essentially of” will be understood to include those elements specifically recited and those additional elements that do not materially affect the basic and novel characteristics of the claimed invention. The phrase “consisting of” excludes any element not specifically specified.

“Complex” refers to a compound formed by the union of one or more electron-rich and electron-poor molecules or atoms capable of independent existence with one or more electronically poor molecules or atoms, each of which is also capable of independent existence.

“Ligand” refers to a species that interacts in some fashion with another species. In one example, a ligand may be a Lewis base that is capable of forming a coordinate bond with a Lewis Acid. In other examples, a ligand is a species, often organic, that forms a coordinate bond with a metal ion. Ligands, when coordinated to a metal ion, may have a variety of binding modes know to those of skill in the art, which include, for example, terminal (i.e., bound to a single metal ion) and bridging (i.e., one atom of the Lewis base bound to more than one metal ion).

“Chelate” or “chelating agent” refers to a molecule, often an organic one, and often a Lewis base, having two or more unshared electron pairs available for donation to a metal ion. The metal ion is usually coordinated by two or more electron pairs to the chelating agent. The terms, “bidentate chelating agent”, “tridentate chelating agent”, and “tetradentate chelating agent” refer to chelating agents having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible.

“Radionuclide” refers to molecule that is capable of generating a detectable image that can be detected either by the naked eye or using an appropriate instrument, e.g. positron emission tomography (PET), and single photon emission tomography (SPECT). Radionuclides useful within the present disclosure include penetrating photon emitters including gamma emitters and X-ray emitters. These rays accompany nuclear transformation such as electron capture, beta emission and isomeric transition. Radionuclides useful include those with photons between 80 and 400 keV and positron producers, 511 keV annihilation photons and acceptable radiation doses due to absorbed photons, particles and half life. Radionuclides include radioactive isotopes of an element. Examples of radionuclides include ¹²³I, ¹²⁵I, ^{99m}Tc, ¹⁸F, ⁶⁸Ga, ⁶²Cu, ¹¹¹In, ¹³¹I, ¹⁸⁶Re, ¹⁸⁸Re, ⁹⁰Y, ²¹²Bi, ²¹¹At, ⁸⁹Sr, ¹⁶⁶Ho, ¹⁵³Sm, ⁶⁷Cu, ⁶⁴Cu, ¹⁰⁰Pd, ²¹²Pb, ¹⁰⁹Pd, ⁶⁷Ga, ⁹⁴Tc, ¹⁰⁵Rh, ⁹⁵Ru, ¹⁷⁷Lu, ¹⁷⁰Lu ¹¹C, and ⁷⁶Br.

“Coordination” refers to an interaction in which one multi-electron pair donor coordinatively bonds (is “coordinated”) to one metal ion.

“Tether” refers to a chemical linking moiety between a metal ion center and another chemical moiety.

“Lewis base” and “Lewis basic” are art-recognized and generally refer to a chemical moiety capable of donating a pair of electrons under certain reaction conditions. It may be possible to characterize a Lewis base as donating a single electron in certain complexes, depending on the identity of the Lewis base and the metal ion, but for most purposes, however, a Lewis base is best understood as a two electron donor. Examples of Lewis basic moieties include uncharged compounds such as alcohols, thiols, and amines, and charged moieties such as alkoxides, thiolates, carbanions, and a variety of other organic anions. In certain examples, a Lewis base may consist of a single atom, such as oxide (O₂⁻). In certain, less common circumstances, a Lewis base or ligand may be positively charged. A Lewis base, when coordinated to a metal ion, is often referred to as a ligand. Further description of ligands relevant to the present invention is presented herein.

In general, “substituted” refers to a group, as defined below (e.g., an alkyl or aryl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls(oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Alkyl groups include straight chain and branched alkyl groups having from 1 to 20 carbon atoms or, in some embodiments, from 1 to 12, 1 to 8, 1 to 6, or 1 to 4 carbon atoms. Alkyl groups further include cycloalkyl groups. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. Where the term haloalkyl is used, the alkyl group is substituted with one or more halogen atoms.

Alkenyl groups include straight and branched chain alkyl and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, 5 to 20 carbon atoms, 5 to 10 carbon atoms, or even 5, 6, 7, or 8 carbon atoms. Examples include, but are not limited to vinyl,

allyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above. Included within this term are the cis and trans isomers or mixtures of these isomers.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8, 2 to 6, or 2 to 4 carbon atoms. Examples include, but are not limited to —C≡CH (acetylenyl), —C≡C(CH₃), —C≡C(CH₂CH₃), —CH₂C≡CH (propargyl), —CH₂C≡C(CH₃), and —CH₂C≡C(CH₂CH₃), among others. Representative substituted alkynyl groups may be mono-substituted or substituted more than once, such as, but not limited to, mono-, di- or tri-substituted with substituents such as those listed above. In some embodiments, the alkynyl groups include that having more than one carbon-carbon triple bond.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Amino acid” refers to all compounds, whether natural or synthetic, which include both an amino functionality and an acid functionality, including amino acid analogs and derivatives.

“Carboxyl” refers to moieties as may be represented by the general formulas:

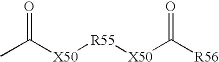

wherein X50 is a bond or represents an oxygen or a sulfur, and R55 and R56 represents a hydrogen, an alkyl, an alkenyl, or a pharmaceutically acceptable salt. Where X50 is an oxygen and R55 or R56 is not hydrogen, the formula represents an “ester.” Where X50 is an oxygen, and R55 is as defined above, the moiety is referred to herein as a carboxyl group, and particularly when R55 is a hydrogen, the formula represents a “carboxylic acid.” Where X50 is an oxygen, and R56 is hydrogen, the formula represents a “formate.” In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

“Sulfonamide” refers to a moiety that may be represented by the general formula:

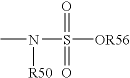

in which R55 and R56 are as defined above.

“Amino” refers to the group —NH₂. “Cyano” refers to the group —CN. “Halo” or “halogen” refers to fluoro, chloro, bromo and iodo and preferably is fluoro or chloro. “Carbonyl” refers to the divalent group —C(O)— which is equivalent to —C(═O)—. “Nitro” refers to the group —NO₂. “Oxo” refers to the atom (═O). “Sulfonyl” refers to the divalent group —S(O)₂—. “Thiol” refers to the group —SH. “Thiocarbonyl” refers to the divalent group —C(S)— which is equivalent to —C(═S)—. “Hydroxy” or “hydroxyl” refers to the group —OH.

“Heteroatom” refers to an atom of any element other than carbon or hydrogen. Exemplary heteroatoms are boron, nitrogen, oxygen, phosphorus, sulfur and selenium.

“Haloalkyl” refers to alkyl groups substituted with 1 to 5, 1 to 3, or 1 to 2 halo groups, wherein alkyl and halo are as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” refers to the group —C(O)NR^{x}R^{y} where R^{x} and R^{y} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R^{x} and R^{y} are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR^{x}R^{y} where R^{x} and R^{y} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R^{x} and R^{y} are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR^{x}R^{y} where R^{x} and R^{y} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R^{x} and R^{y} are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above. In one embodiment, the nitrogen and/or the sulfur ring atom(s) of the heteroaryl group are optionally oxidized to provide for the N-oxide (N→O), sulfinyl, or sulfonyl moieties.

Heterocyclyl groups include aromatic (also referred to as heteroaryl) and non-aromatic ring compounds containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties. Heterocyclyl groups encompass unsaturated, partially saturated and saturated ring systems, such as, for example, imidazolyl, imidazolinyl and imidazolidinyl groups. The phrase “heterocyclyl group” includes fused ring species including those including fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

“Stereoisomer” or “stereoisomers” refer to compounds that differ in the chirality of one or more stereocenters. Stereoisomers include enantiomers and diastereomers.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P.G.M. Protective Groups in Organic Synthesis, 2nd ed.; Wiley: New York, 1991).

“Pharmaceutically acceptable salts” refers to relatively non-toxic, inorganic and organic acid addition salts of compositions, including without limitation, analgesic agents, therapeutic agents, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The phrase “pharmaceutically acceptable carrier” is art-recognized, and includes, for example, pharmaceutically acceptable materials, compositions or vehicles, such as a liquid or solid filler, diluent, solvent or encapsulating material, involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrase “therapeutically effective amount” refers to the amount of a substance to bring about a therapeutic response. For example, a therapeutically effective, VLA-4 inhibitive amount of a complex or compound of formula I, II, III, or IV. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to: the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the use of concomitant medication; and other relevant circumstances.

“Subject” refers to mammals and includes humans and non-human mammals.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

The invention is generally based on identification of compounds that afford affinity and/or selectivity for VLA-4. In one aspect, peptide inhibitors of VLA-4 are incorporated with a chelate-metallic moiety comprising a radionuclide to form a complex. The radionuclide incorporated into the complex is adapted for radioimaging and/or radiotherapy.

In one aspect, a complex of formula I, its stereoisomer or pharmaceutically acceptable salt is provided:

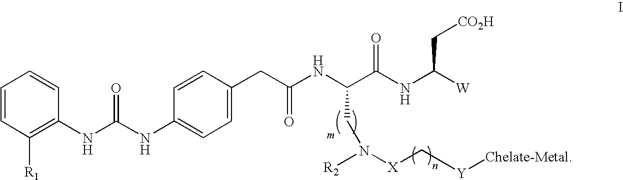

In formula I, W is a group selected from the group consisting of

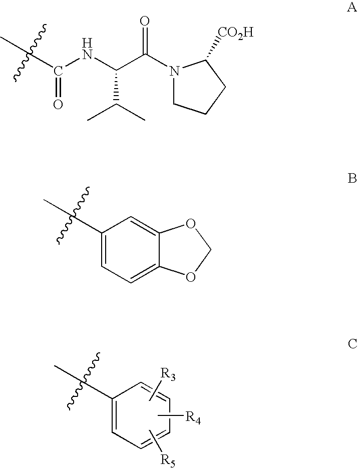

R₁ is hydrogen, alkyl or alkoxy; R₂ is a bond, hydrogen or alkyl; R₃, R₄ and R₅ are independently hydrogen, iodine, alkyl, alkoxy, hydroxyl, amino, aminoalkyl, dialkylamino, or carboxyl; X is a bond, C═O, O═C—O, or CH₂; Y is a bond, CH₂, or O; m is an integer ranging from 1 to 6; n is an integer ranging from 0 to 6; Metal represents a metallic moiety comprising a radionuclide; and Chelate represents a chelating moiety that coordinates with said radionuclide to form the complex. In some embodiments, the radionuclide is selected from the group consisting of technetium, rhenium, yttrium, indium, gallium, gadolinium, and copper.

In some embodiments, the Chelate is selected from the group consisting of 1,4,7,10-tetraazacyclododecane-1,4,7,10-tetraacetic acid (DOTA), diethylenetriaminepentaacetic acid (DTPA), pyridylmethylene amine (PDA), quinolinemethylene amine, isoquinoline amine, pyridine-2-ylmethylamino acetic acid (PAMA), isoquinolin-3-ylmethylamino acetic acid, thiazol-2-ylmethyl amine, thiazol-2-ylmethylamino acetic acid, N-methylimidazole(methylene)amine, N-methylimidazole(methylene)amino acetic acid NOTA, Hynic, MAG3, N₂S₂, MAMA and DADT.

In some embodiments, the Metal-Chelate moiety is selected from:

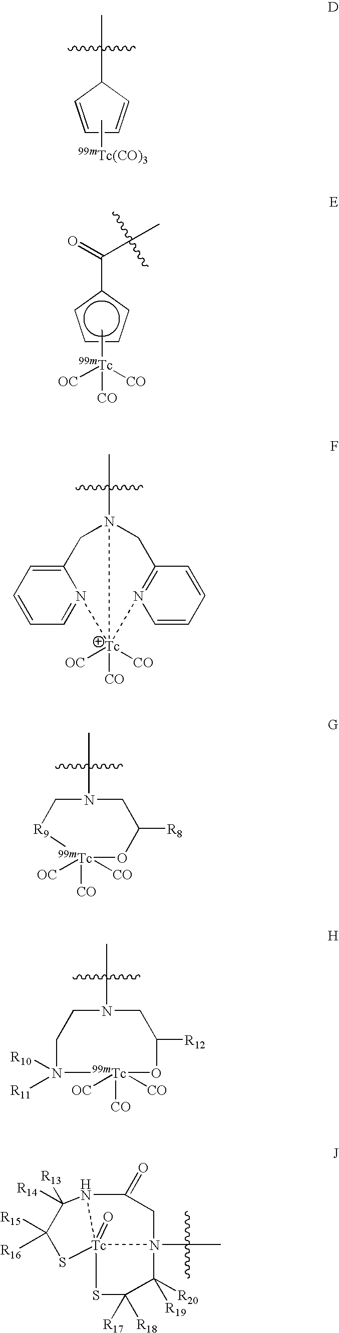

where R₈ is selected from the group consisting of 0, H, OH, alkoxy, or O-alkyl; R₉ is a pharmaceutically acceptable 5 or 6-member heterocyclic ring; R₁₀ and R₁₁ are each independently hydrogen, alkyl, or substituted alkyl; R₁₂ is selected from the group consisting of aryl, alkyl, or heterocycle; and R₁₃, R₁₄, R₁₅, R₁₆, R₁₇, R₁₈, R₁₉, R₂₀ are independently hydrogen or methyl. In some embodiments, the complex of formula I has the structure I-a, I-b, I-c, I-d, I-e, or I-f:

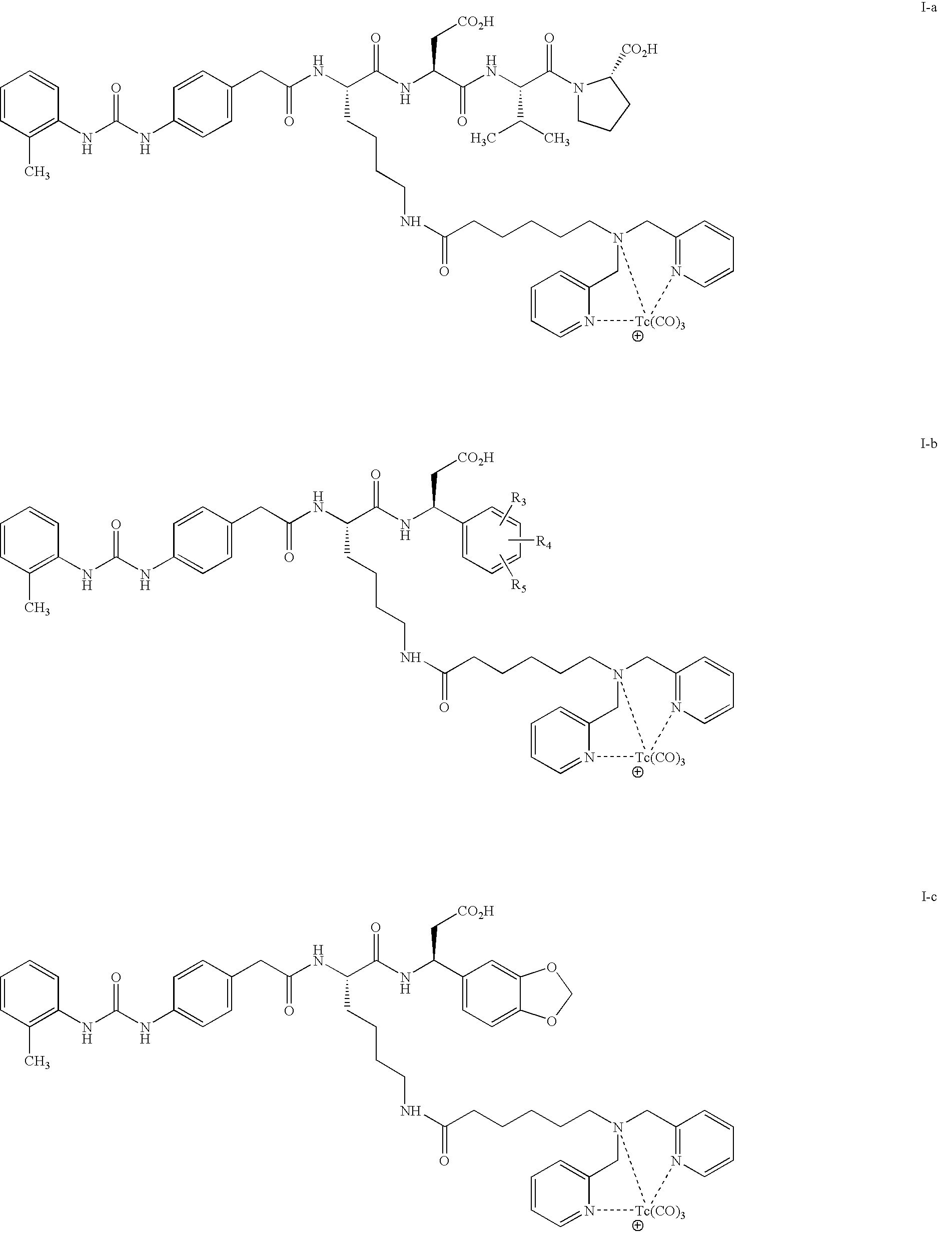

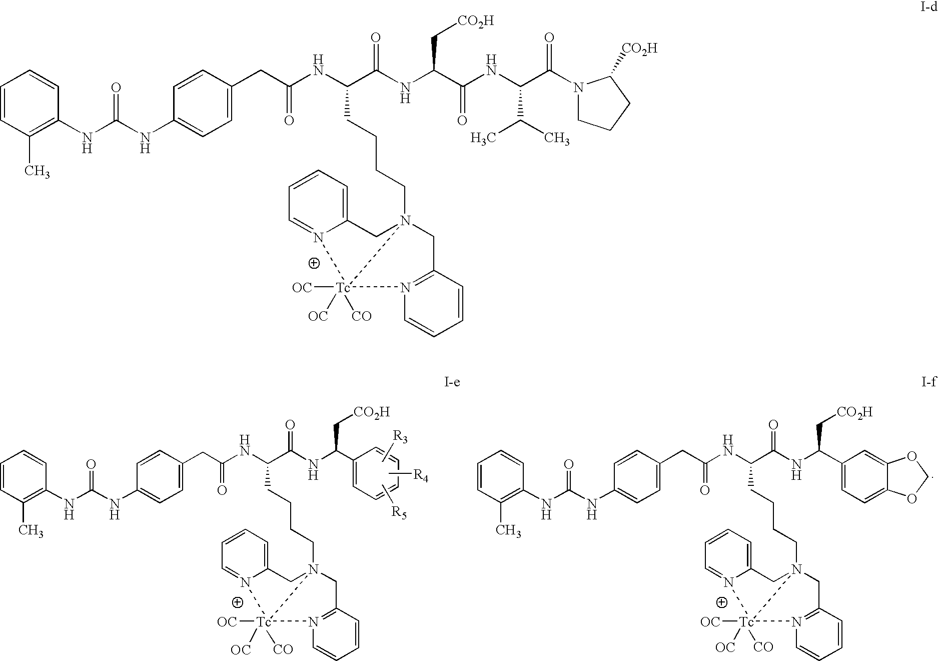

In formulas I-a to I-f, R₃, R₄ and R₅ are independently hydrogen, iodine, alkyl, alkoxy, hydroxyl, amino, aminoalkyl, dialkylamino, or carboxyl.

The complex or compound represented by formula I may be prepared by methods known in the art. In general, the complex represented by formula I may be prepared by incorporating a Metal-Chelate moiety into a compound containing targeting moiety that exhibits selective binding to VLA-4 and a chelate capable of complexing a metal.

By way of example, the Metal-Chelate compounds may be made by Single Amino Acid Chelate (SAAC™) technology, which is described in U.S. Patent Application Publication No. 2003/0235843, the disclosure of which is incorporated herein by reference in its entirety. A variety of structurally diverse molecules can be made using the SAAC technology. The SAAC technology may provide a rapid, high yield, one pot synthesis of mono-, di-, and mixed alkylated amino acid derivatives. The alkylated amino acid derivatives may possess a tridentate chelating moiety distal to an amino acid functionality. The tridentate chelating group allows facile and robust coordination of a metallic moiety or metallic core such as {M(CO)₃}⁺¹ core (M is a radionuclide such as Tc or Re). In some embodiments, a metallic core may be inserted prior to performing standard chemistries, including standard deprotection and peptide cleavage chemistries, without loss of the metal from the SAAC complex. Studies on the coordination chemistry of the {M(CO)₃}⁺¹ core have established that amine, aromatic, heterocyclic, and carboxylate donors provide effective chelating ligands. The tridentate chelate-M(CO)₃ complex provide chemical inertness and a broad utility of the amino acid functionality. Various tridentate chelating moieties can be made so as to alter the charge, hydrophobicity, and distance of the tridentate chelate-M(CO)₃ complex from the functional moiety of the compound. Scheme 1 illustrate preparation of alkylated SAAC molecules by direct reductive N-alkylations of t-butyloxycarbonyl (BOC) protected lysine with the desired aldehydes with NaBH(OAc)₃ as the reducing agent.

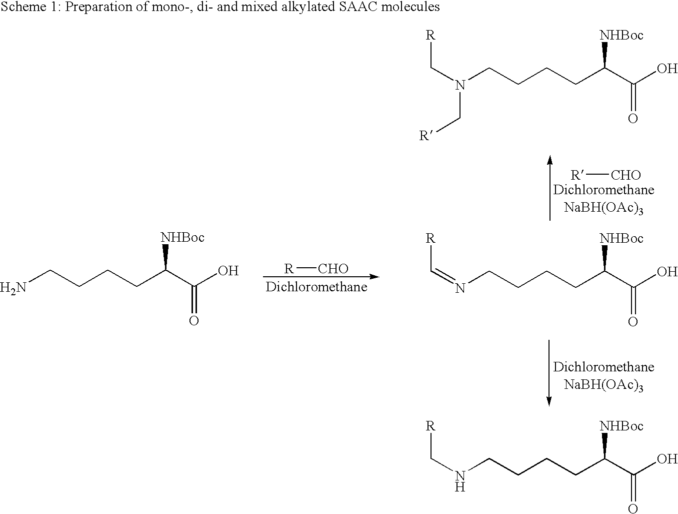

where R and R′ are independently a, b, c, d, e, f, or g:

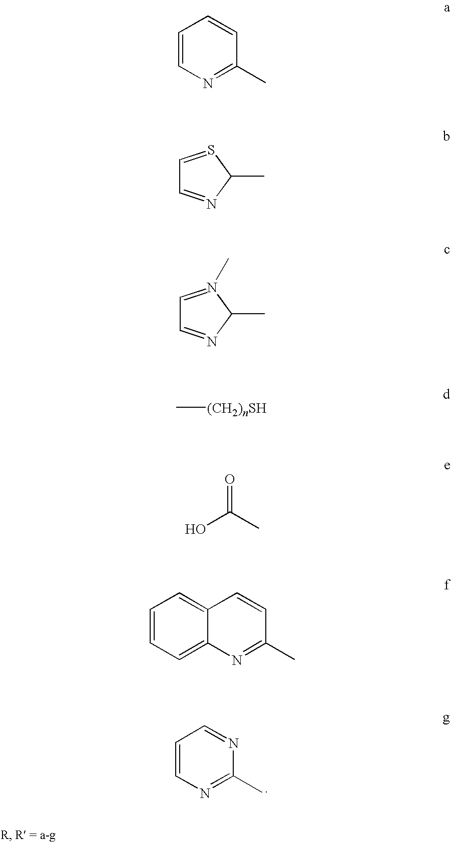

The {M(CO)₃}⁺¹ (M is e.g. Tc or Re) complexes of the bifunctional chelates can be readily prepared from for example (Et₃NH)[Tc(CO)₃(H₂O)₃] and (Et₄N)₂[Re(CO)₃Br₃], respectively.

The complex or compound of the invention may be used in accordance with the methods described herein by those skilled in the art, e.g., by specialists in nuclear medicine, for diagnostic imaging of tissue which expresses VLA-4, and therapeutic treatment of diseases which are characterized by expressing VLA-4.

The complex or compound of the invention may be used in the following manner. An effective amount of the compound (from 1 to 50 mCi) may be combined with a pharmaceutically acceptable carrier for use in imaging studies. In accordance with the invention, “an effective amount” of the compound is defined as an amount sufficient to yield an acceptable image using equipment which is available for clinical use. An effective amount of the complex may be administered in more than one injection. Effective amounts of the complex will vary according to factors such as the degree of susceptibility of the individual, the age, sex, and weight of the individual, idiosyncratic responses of the individual and dosimetry. Effective amounts of the complex will also vary according to instrument and film-related factors. Optimization of such factors is well within the level of skill of a person skilled in the art.

As used herein, the pharmaceutically acceptable carrier includes any and all solvents, dispersion media, coatings, antibacterial and antifungal agents, isotonic agents, absorption delaying agents, and the like. The use of such media and agents for pharmaceutically active substances is well known in the art. The complex or compound may be administered to an individual in an appropriate diluent or adjuvant, or in an appropriate carrier such as human serum albumin or liposomes. Supplementary active compounds can also be used with the complex. Pharmaceutically acceptable diluents include saline and aqueous buffer solutions. Adjuvants contemplated herein include resorcinols, non-ionic surfactants such as polyoxyethylene oleyl ether and hexadecyl polyethylene ether.

In one embodiment, the complex or compound is administered parenterally as injections (intravenous, intramuscular or subcutaneous). The complex or compound may be formulated as a sterile, pyrogen-free, parenterally acceptable aqueous solution. The preparation of such parenterally acceptable solutions, having due regard to pH, isotonicity, stability, and the like, is within the skill in the art. Certain pharmaceutical compositions suitable for parenteral administration comprise one or more imaging agents in combination with one or more pharmaceutically acceptable sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. A formulation for injection should contain, in addition to the cardiovascular imaging agent, an isotonic vehicle such as sodium chloride solution, Ringer's solution, dextrose solution, dextrose and sodium chloride solution, lactated Ringer's solution, dextran solution, sorbitol solution, a solution containing polyvinyl alcohol, or an osmotically balanced solution comprising a surfactant and a viscosity-enhancing agent, or other vehicle as known in the art. The formulation used in the present invention may also contain stabilizers, preservatives, buffers, antioxidants, or other additives known to those skilled in the art.

The amount of the complex or compound used for diagnostic or therapeutic purposes will depend upon the nature and severity of the condition being treated, on the nature of therapeutic treatments which the patient has undergone, and on the idiosyncratic responses of the patient. Ultimately, the attending physician will decide the amount of complex or compound to administer to each individual patient and the duration of the imaging study.

In another aspect, the invention provides a kit for imaging which comprises one or more of the complex or compound described above, in combination with a pharmaceutically acceptable solution containing a carrier such as human serum albumin or an auxiliary molecule such as mannitol or gluconate. Human serum albumin for use in the kit of the invention may be made in any way, for example, through purification of the protein from human serum or through recombinant expression of a vector containing a gene encoding human serum albumin. Other substances may also be used as carriers, for example, detergents, dilute alcohols, carbohydrates, and the like. In one embodiment, a kit according to the invention may contain from about 1 to about 30 mCi of a complex or compound. In another embodiment, a kit may contain the unlabeled fatty acid stereoisomer which has been covalently or non-covalently combined with a chelating agent, and an auxiliary molecule such as mannitol, gluconate, and the like. The unlabeled fatty acid stereoisomer/chelating agent may be provided in solution or in lyophilized form. The kits may also include other components which facilitate practice of the methods of the invention. For example, buffers, syringes, film, instructions, and the like may optionally be included as components of the kits of the disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present embodiments, thus generally described, will be understood more readily by reference to the following examples, which are provided by way of illustration and are not intended to be limiting of the present technology in any way.

### EXAMPLES

The following examples are provided to illustrate certain aspects of the present invention and to aid those skilled in the art in practicing the invention. These examples are not intended to limit the scope of the invention. In the examples, the following abbreviations are used:

TFA: trifluoroacetic acid

EDC: 1-Ethyl-3-(3-dimethyllaminopropyl)carbodiimide hydrochloride.

BOC: Di-tert-butyl Dicarbonate

TEA: triethylamine

CBZ: Carbobenzyloxy

The following exemplary compound can be synthesized from intermediate (1). Intermediate (1) can be prepared by modifications to methods described in J. Med. Chem. 1999, 42, 920, the disclosure of which is incorporated herein by reference. Coupling of BOC valine (2) with proline methyl ester hydrochloride salt (3) utilizing EDC/HOBt, followed by cleavage of the BOC protecting group with TFA affords dipeptide TFA salt (4). Repeating the coupling deprotection sequence with TFA salt (4) and protected aspartic acid derivative (5) affords the protected tripeptide TFA salt (6). Coupling of (6) to protected lysine derivative (7) followed by deprotection affords tetrapeptide TFA salt (8) which is then coupled to diphenylurea activated ester (9). Removal of CBZ protecting group by hydrogenolysis affords intermediate (1).

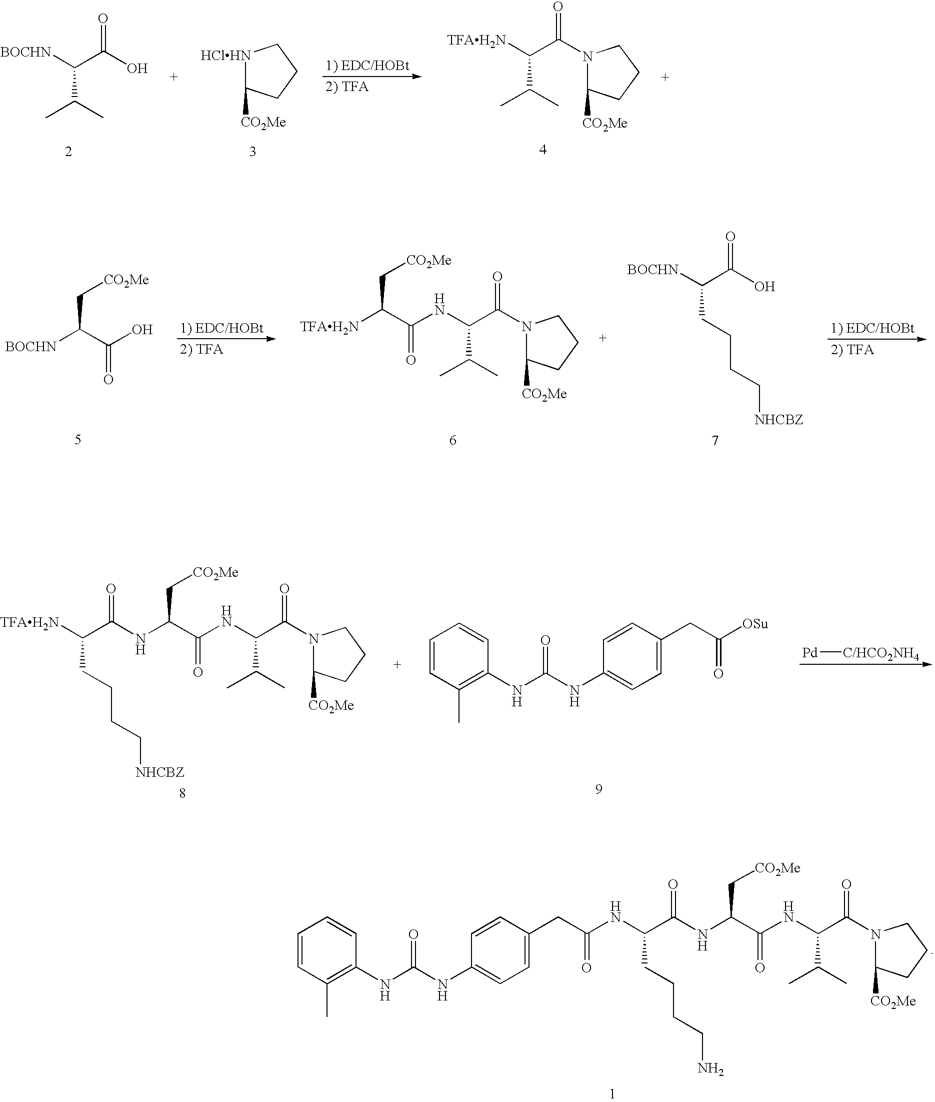

Intermediate (1) can be derivatized directly by reductive amination with pyridine-2-carboxaldehyde which after hydrolysis of the methyl ester protecting groups affords ligand (10). Ligand (10) can be utilized directly to form Rhenium complex (12). This complex serves as the cold reference material for the corresponding radioactive ^{99m}Tc analog.

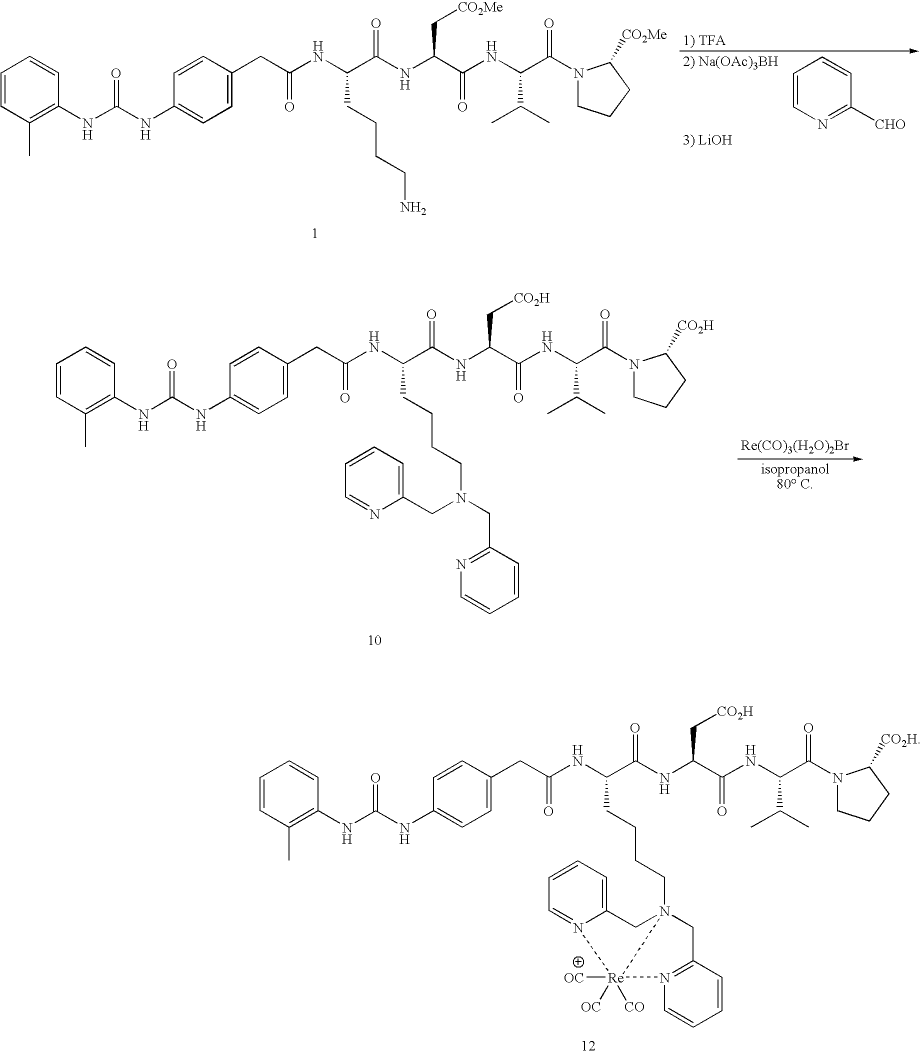

The radioactive ^{99m}Tc analog (13) can be prepared from the free acid utilizing the Isolink kit methodology described below.

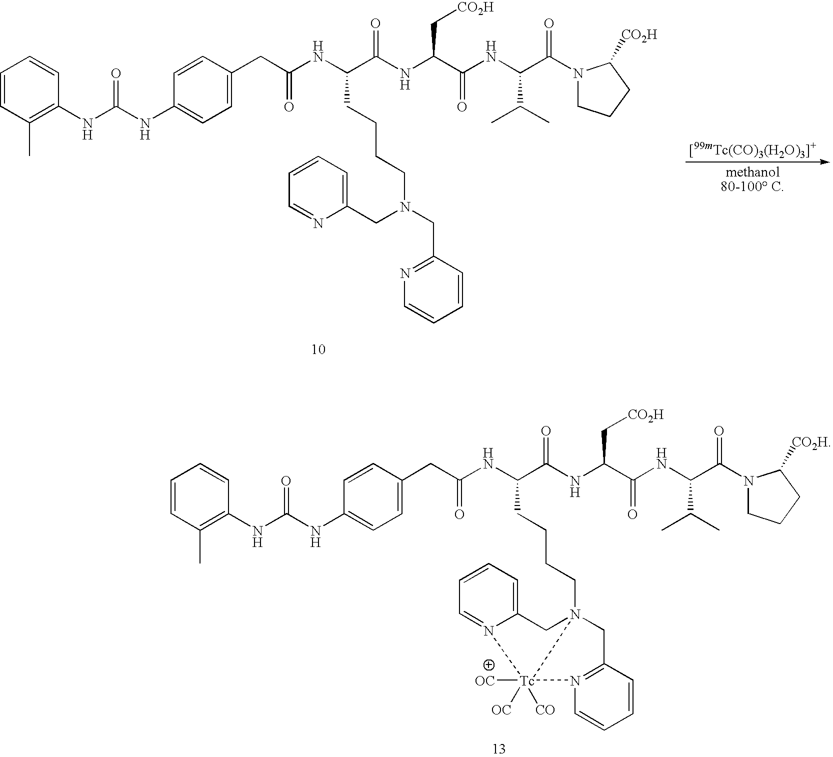

In addition to the hydrophobic cationic complexes (12) and (13) the neutral PAMA analogs can be synthesized from intermediate (1). Reductive amination with one equivalent of pyridine-2-carboxaldehyde affords (14) which after alkylation with the desired bromoacetate such as t-butylbromoacetate give the protected ligand (15). Selective removal of the t-butyl ester protecting group affords (16).

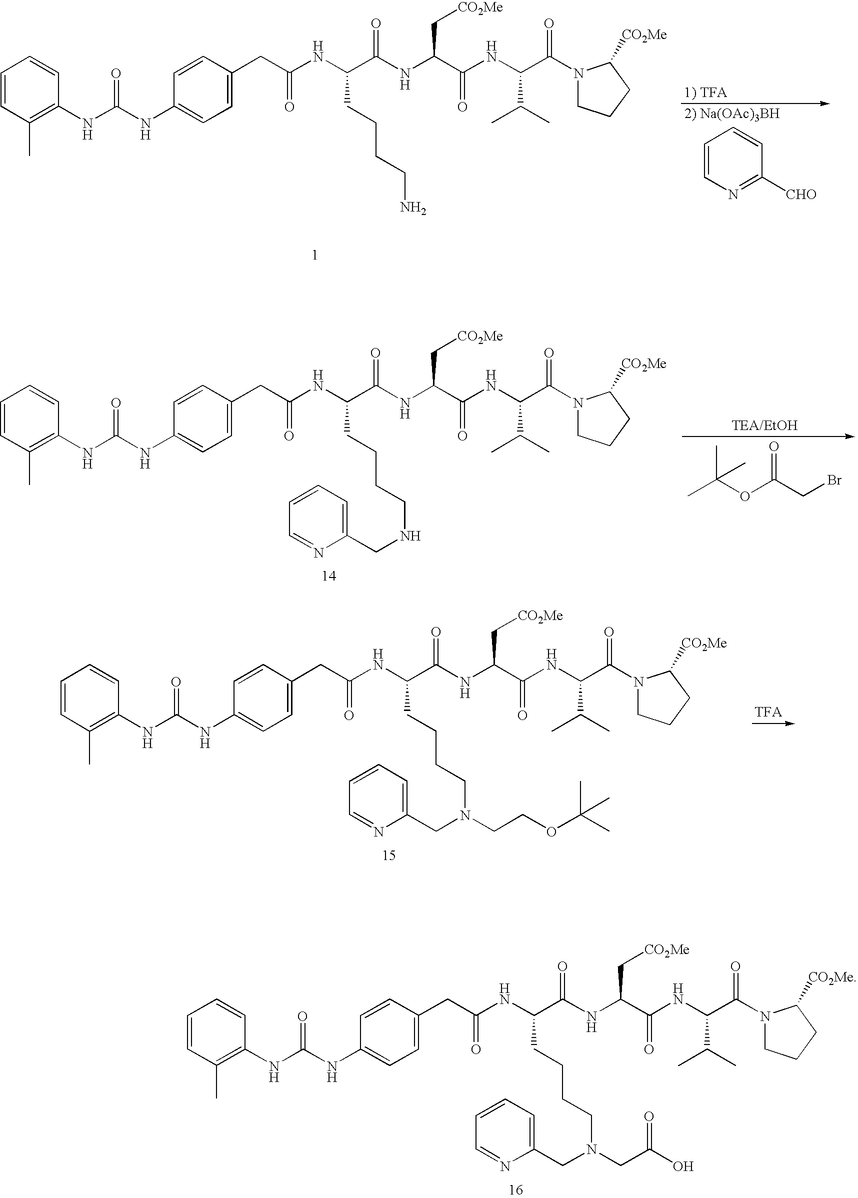

Formation of the rhenium complex (18) can be accomplished in two manners. Intermediate (16) can be complexed and then deprotected to remove the methyl ester protecting groups to afford (18). Alternatively, removal of the methyl ester protecting groups gives (17) which can then be complexed with rhenium to afford the desired complex (18).

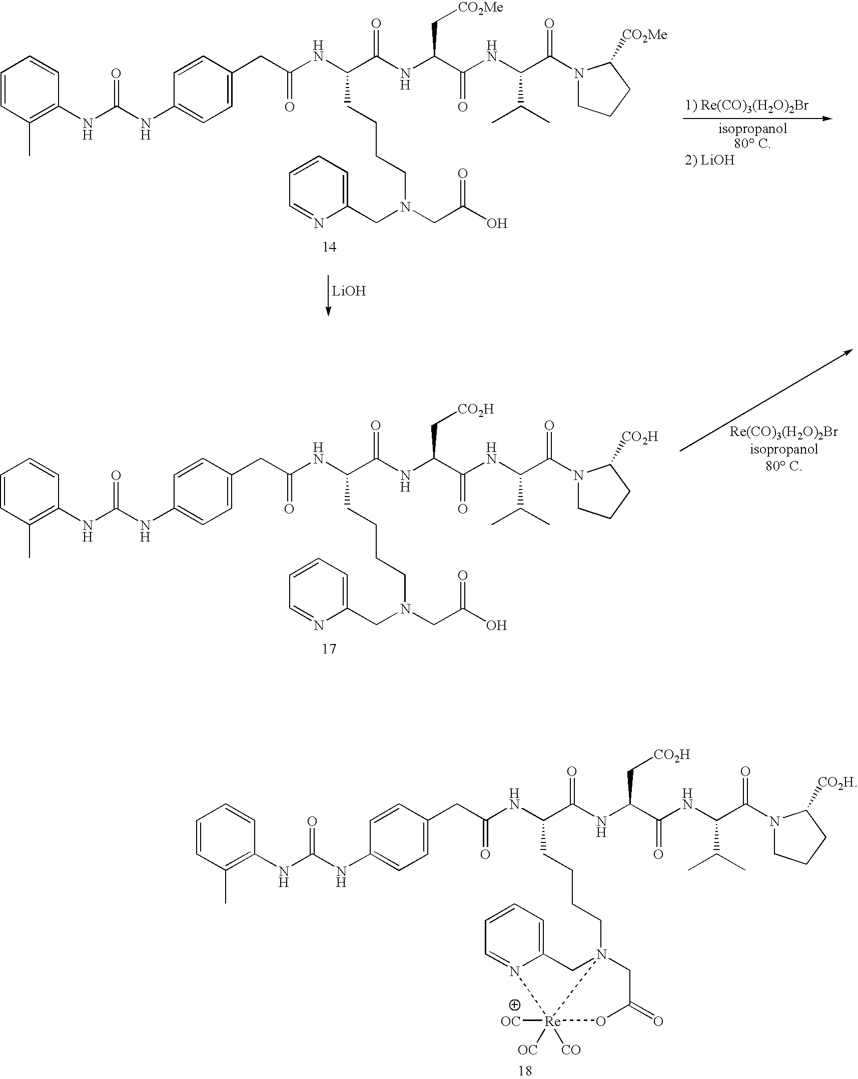

The radioactive ^{99m}Tc analog (19) can be prepared from the deprotected ligand (17).

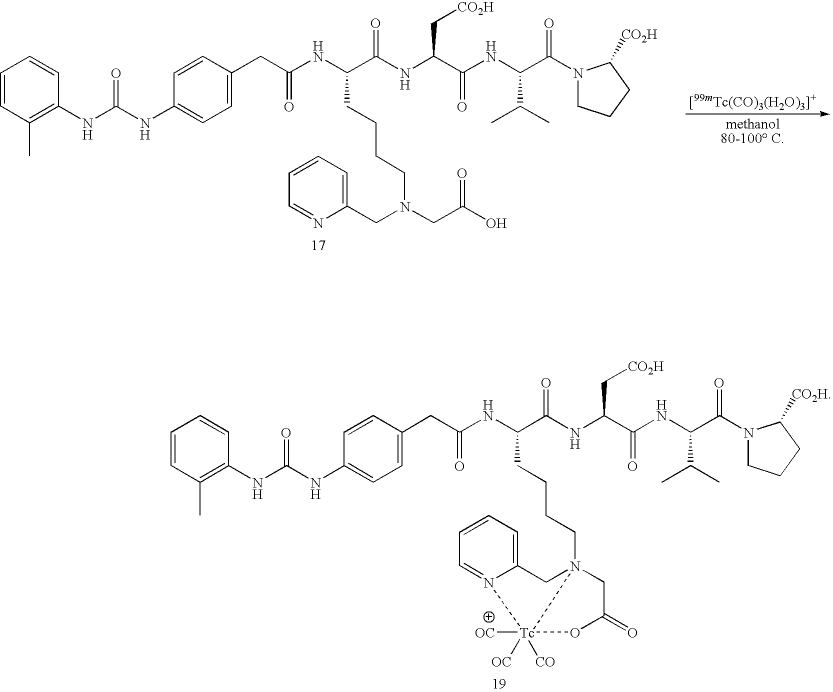

The various analogs that incorporate various linkers off of the side chain lysine amine can also be prepared from intermediate (1). For example the analog with the 6-aminohexanoic acid linker can be prepared by coupling of (1) with bis(pyridine-2-ylmethyl)amino)hexanoic acid which can be readily prepared from 6-aminohexanoic acid (Zubieta et. al, Synthesis 2004, 11, 1759). Hydrolysis of the methyl esters yields the desired ligand (21).

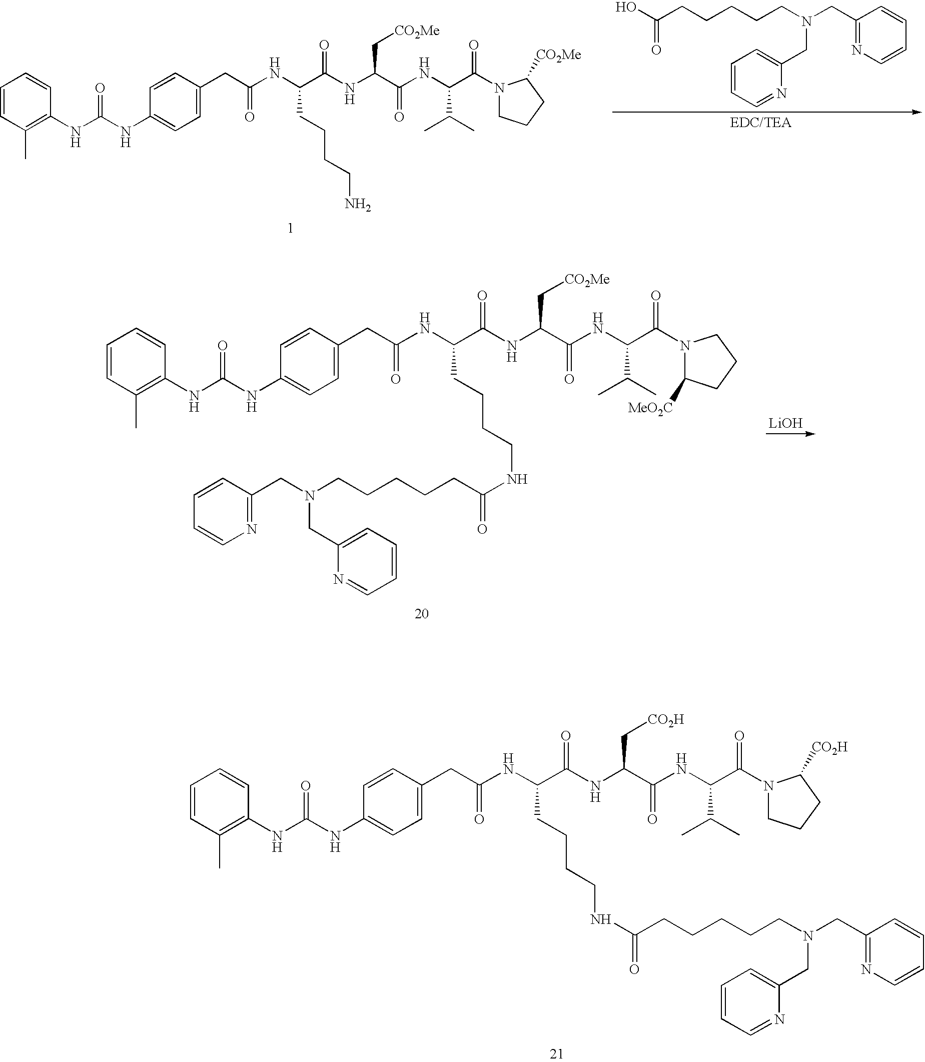

Ligand (21) can be converted to the desired cold rhenium standard (22) as well as the radioactive ^{99m}Tc analog (23).

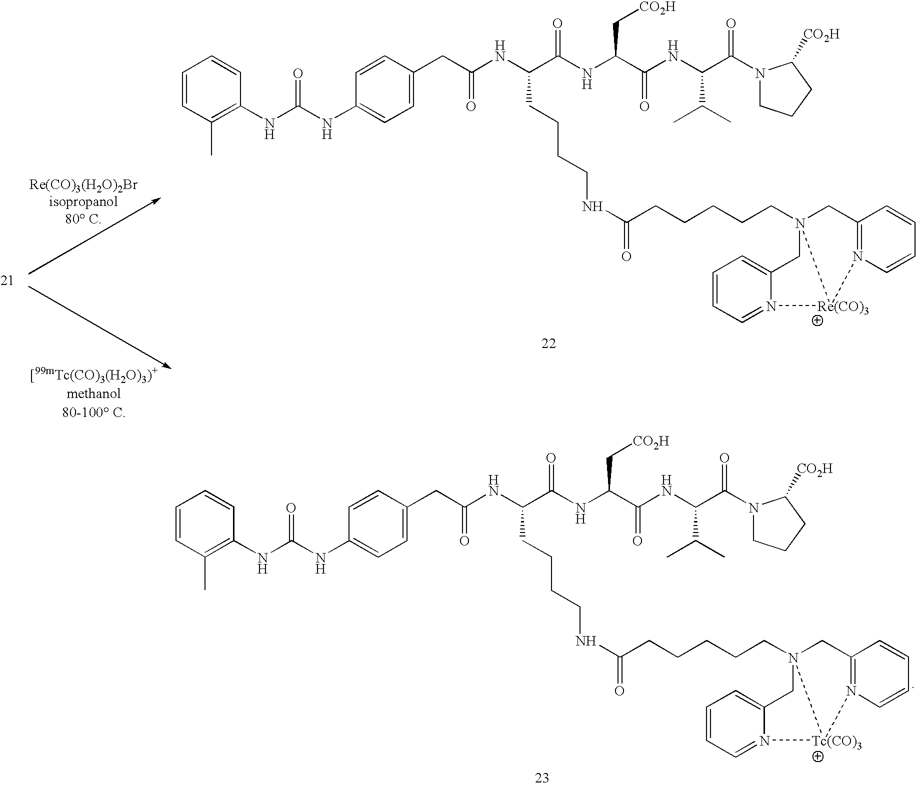

Intermediate (1) can also be readily conjugated to dicarboxylic acids. For example, succinic acid can be conjugated to (1) using succinic anhydride to afford the free acid (24). Coupling of the chelator via a free amine such as (25) to the free acid (24) affords (26) after hydrolysis of the methyl ester protecting groups.

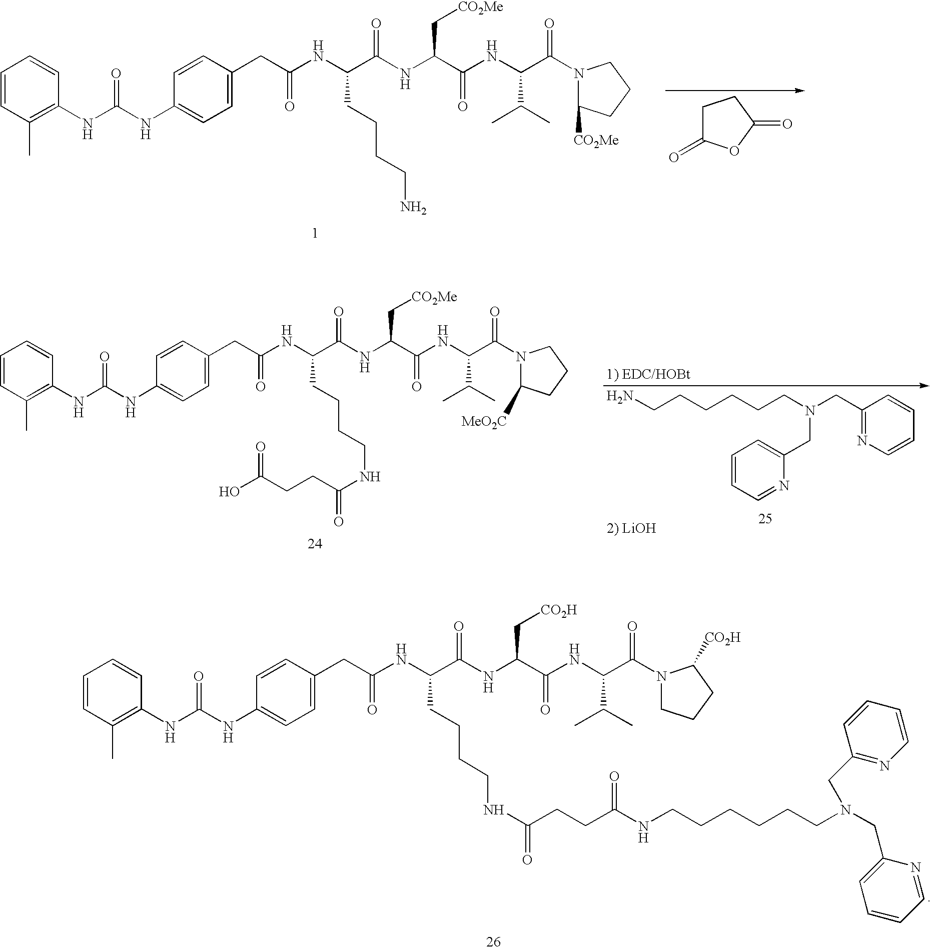

Intermediate (26) can be utilized to synthesis both the rhenium standard (27) as well as the ^{99m}Tc analog (28).

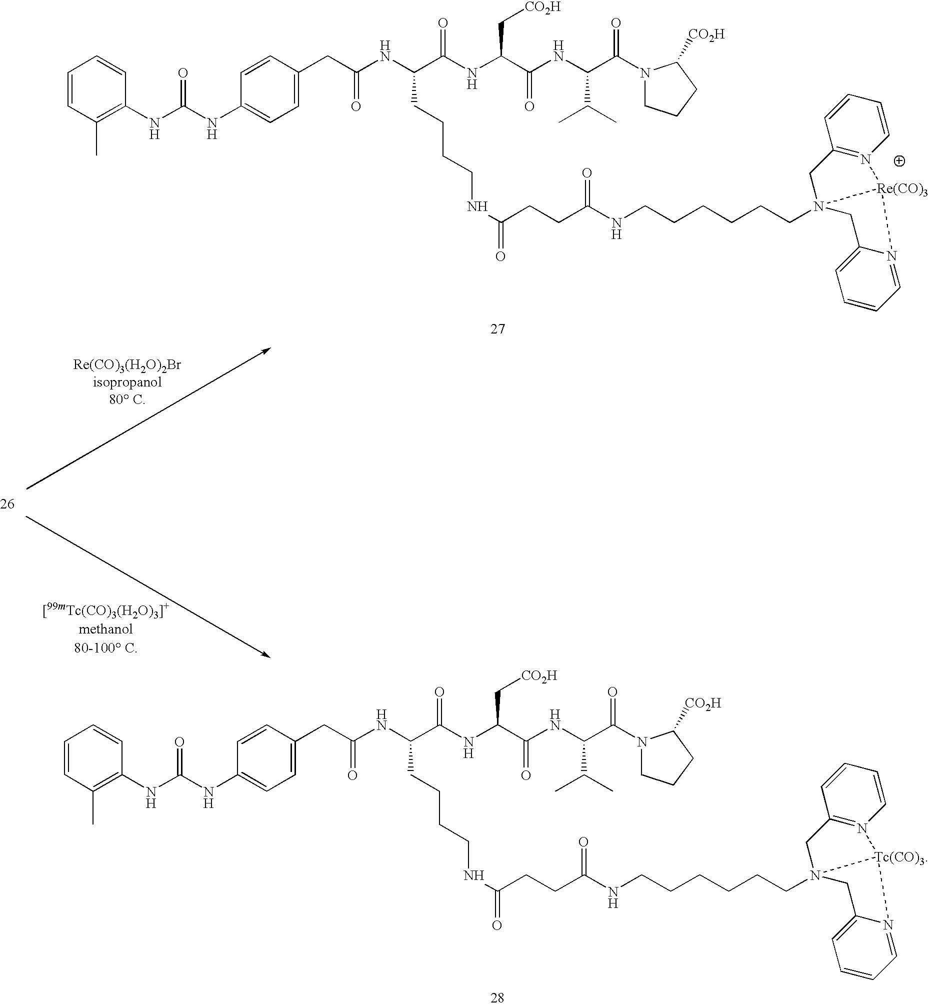

The analogs where the β-aminoacid replaces the aspartic acid-proline and valine residues are prepared as described below. The readily accessible β-amino acids (Davies et. al, Yuki Gosei Kagaku Kyokaishi 1997, 55(1), 42-50), such as the methylenedioxy species (29) can be converted into the protected intermediate (32).

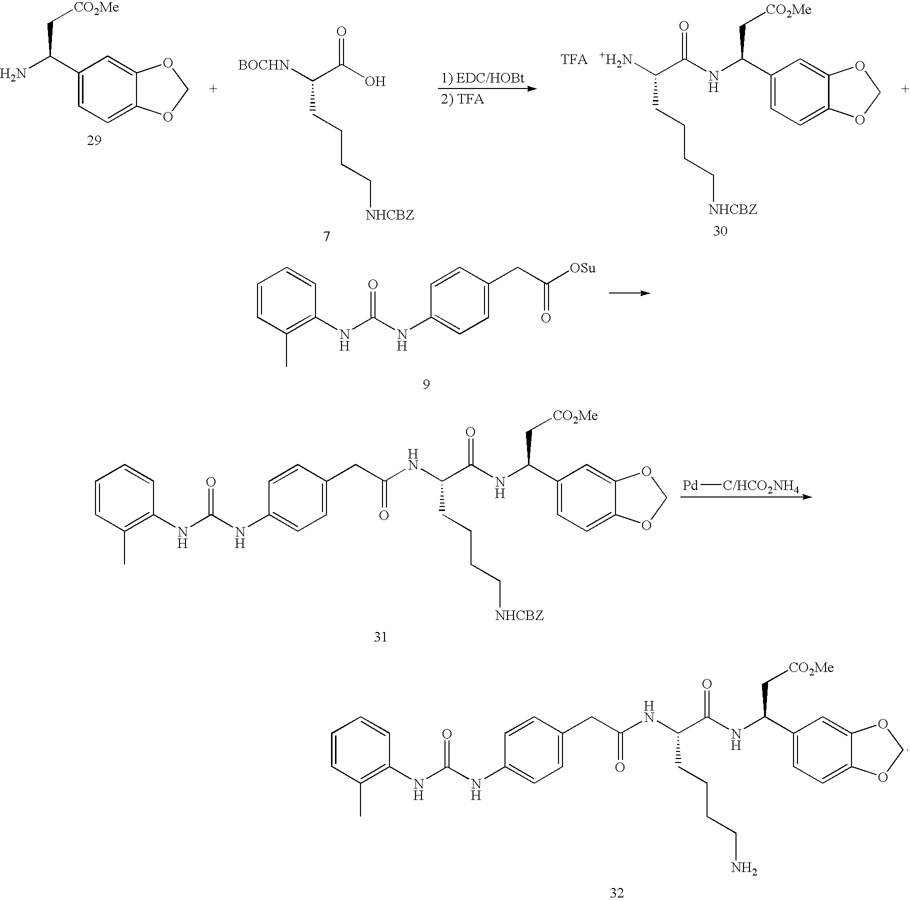

Modification of (32) affords the ligands (33), (34) and (35), utilizing the various methods described above, which can be converted into the corresponding rhenium or technetium analogs.

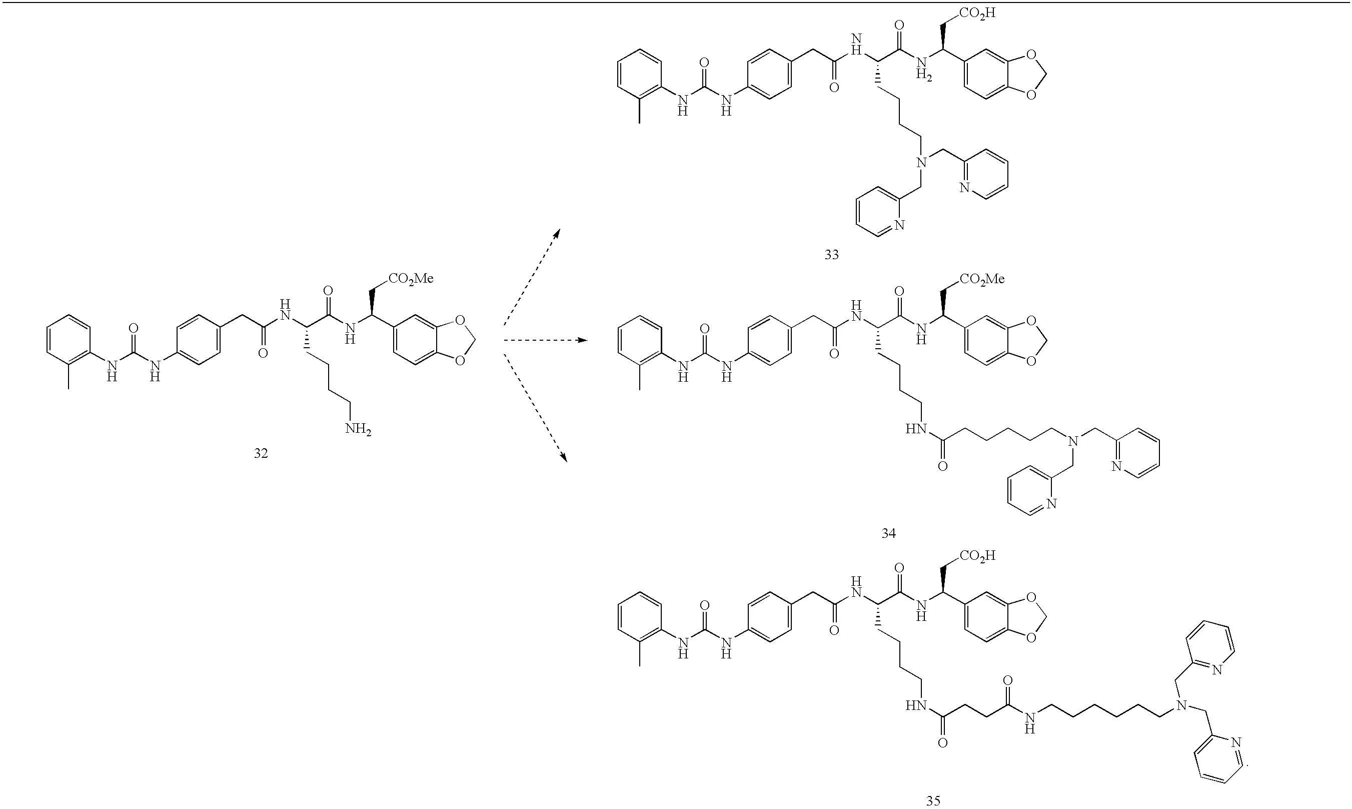

The synthesis of the rhenium analogs (36), (37) and (38) are completed by complexation with Re(CO)₃(H₂O)₂Br to afford the rhenium complexes.

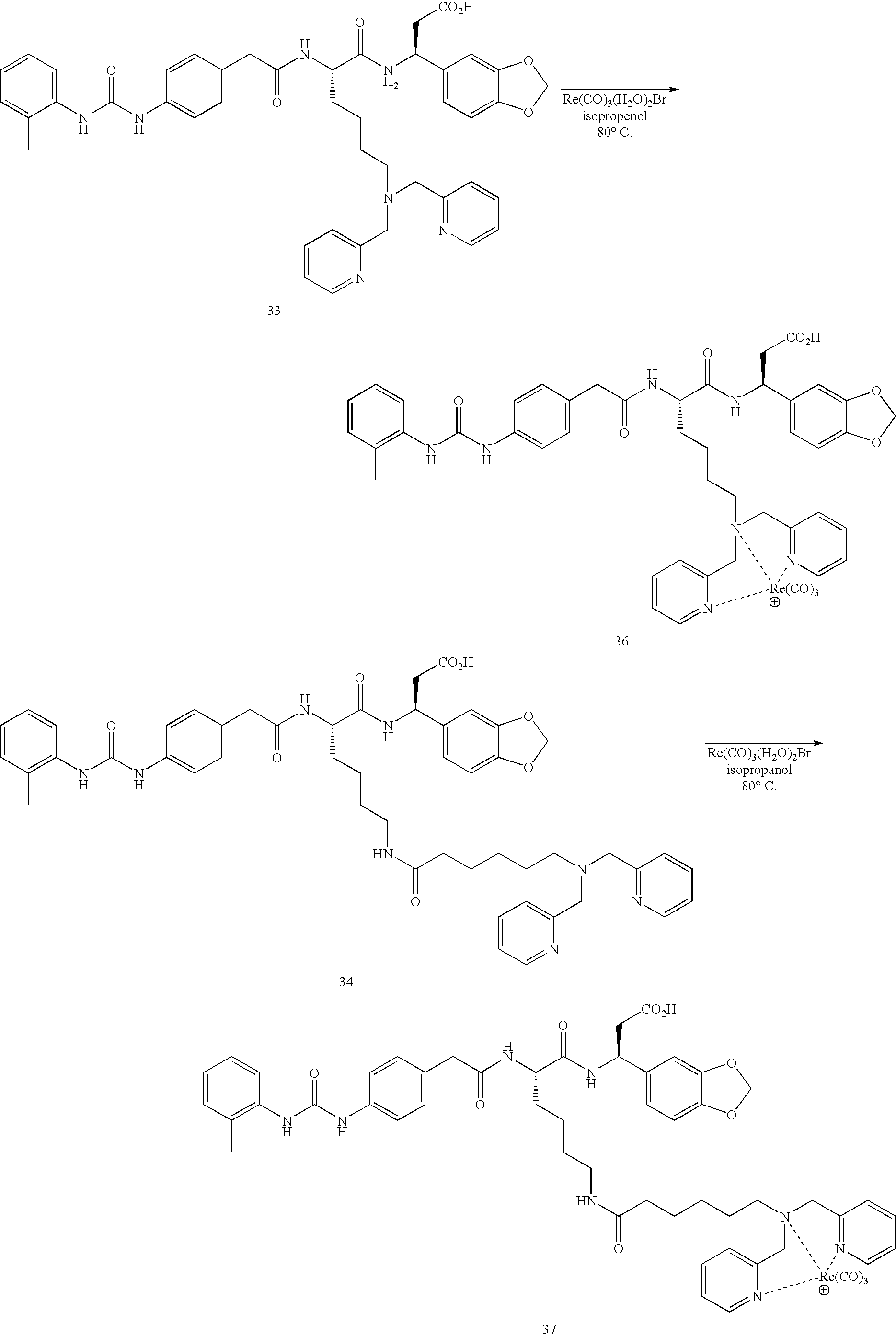

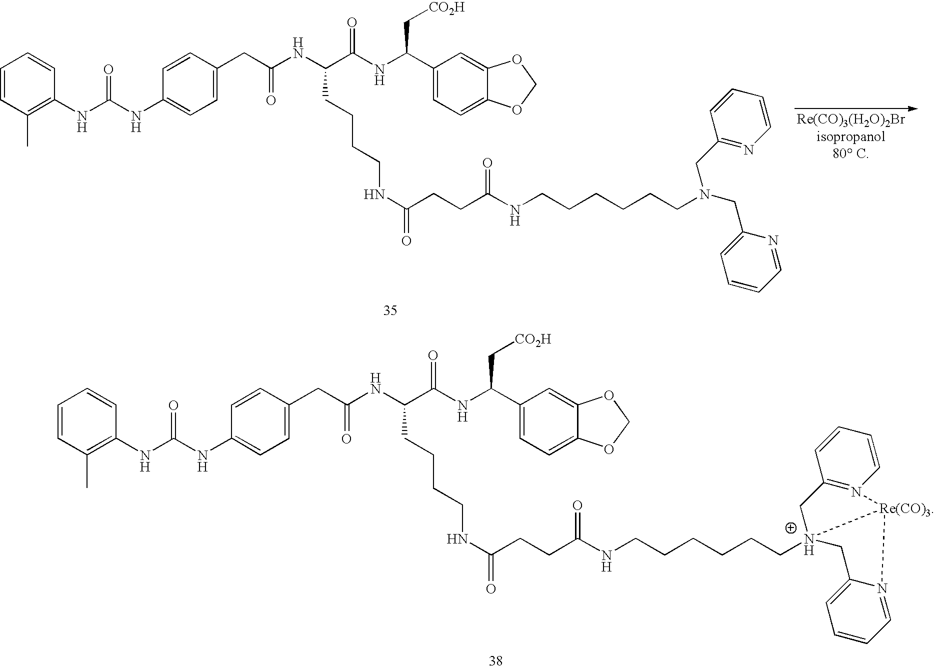

The corresponding radioactive ^{99m}Tc analogs, (39), (40) and (41) can be prepared from free ligands (33), (34) and (35) respectively.

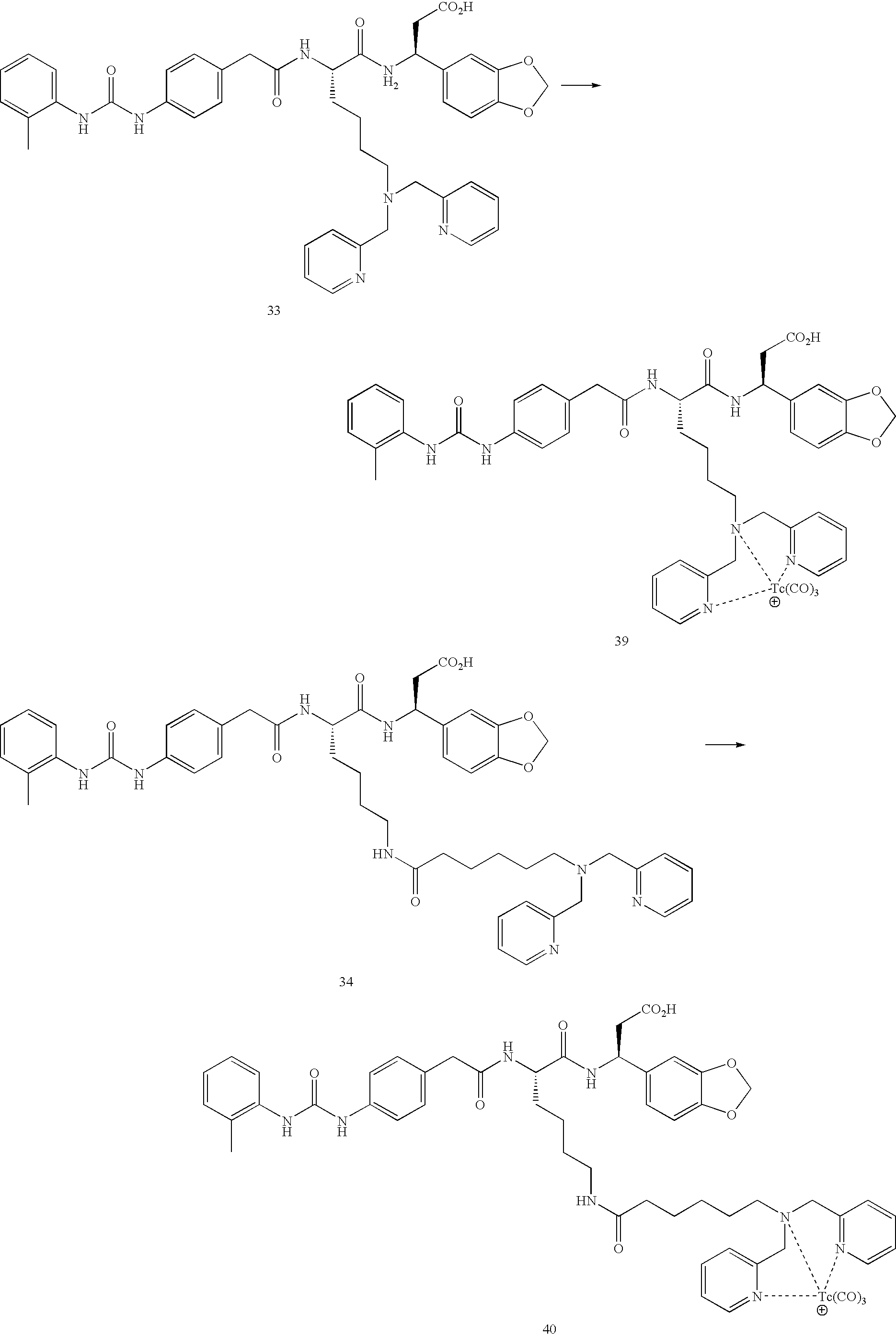

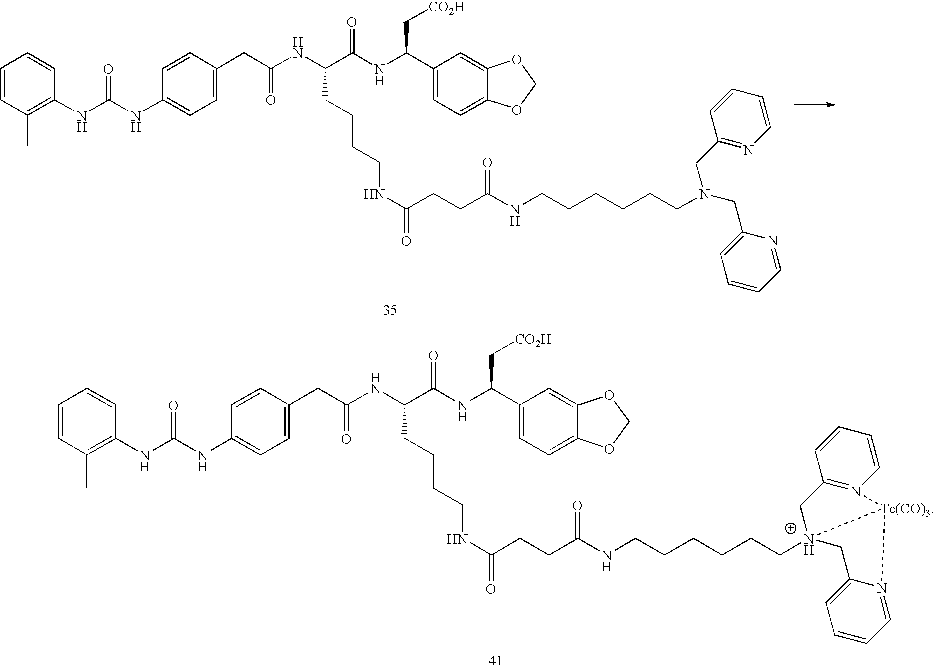

The neutral PAMA ligands of the hexanoic acid linked analogs shown in the scheme below. Intermediate (1) is coupled to the readily available free acid 6-((2-tert-butoxy-2-oxoethyl)(pyridin-2-ylmethyl)amino)hexanoic acid to afford the protected ligand (42) which after deprotection gives the ligand (43).

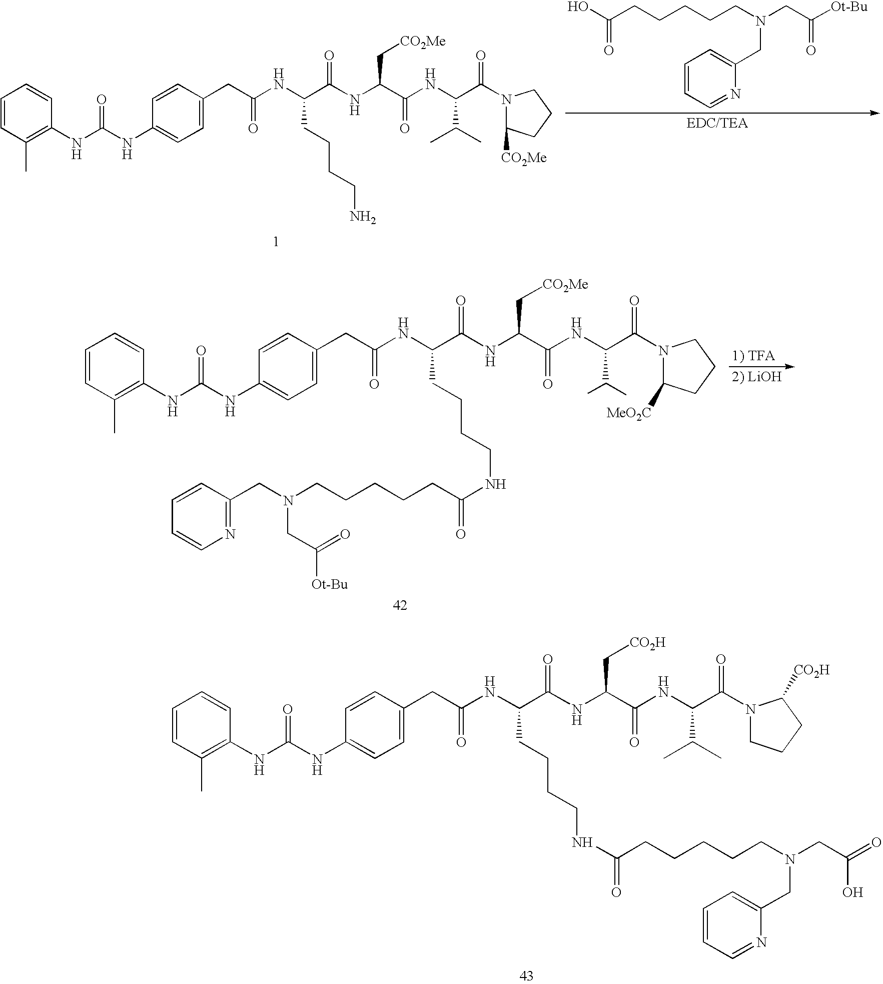

Ligand (43) can be readily converted into the rhenium analog (44) or the ^{99m}Tc analog (45).

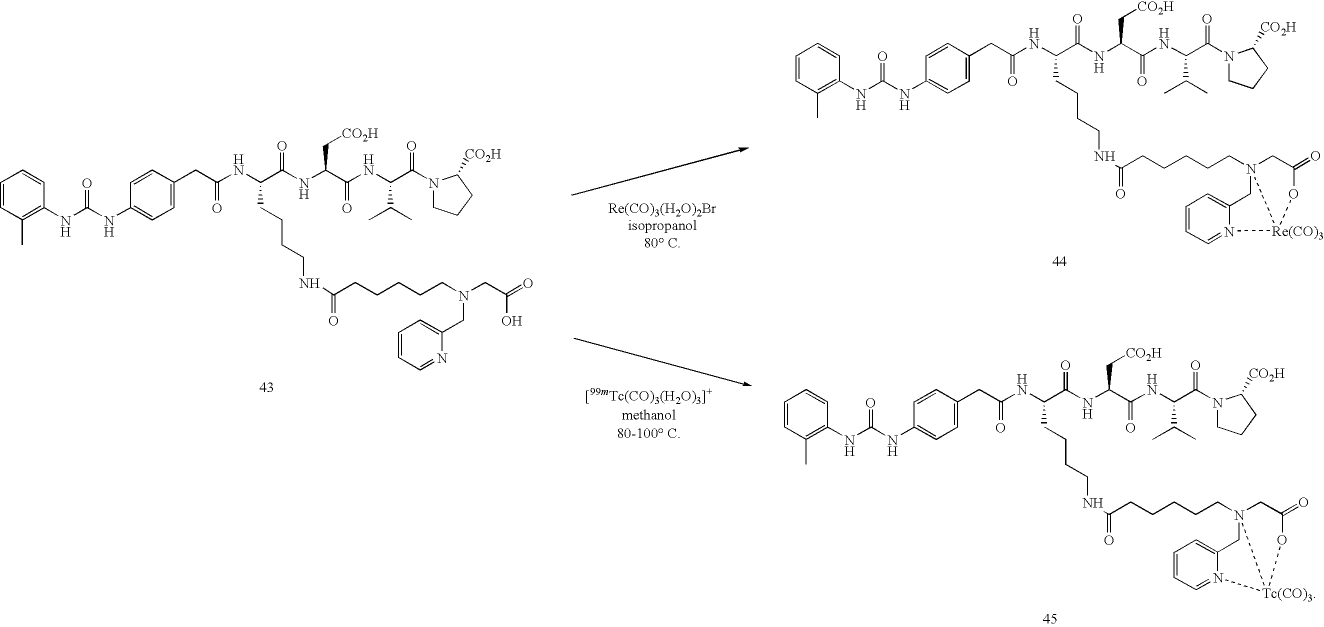

The succinic acid linked PAMA ligand can be prepared from intermediate (24) by coupling to tert-butyl 2-((6-aminohexyl)(pyridin-2-ylmethyl)amino)acetate followed by removal of the t-butyl protecting group and hydrolysis of the methyl esters to give (46).

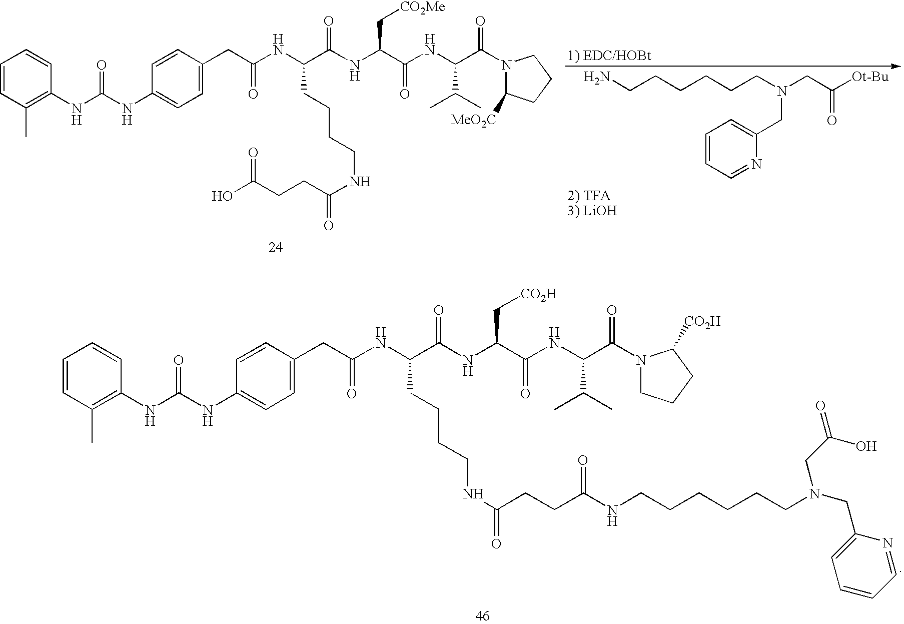

Ligand (46) can be converted to both the cold rhenium analog (47) and the radioactive ^{99m}Tc analog (48).

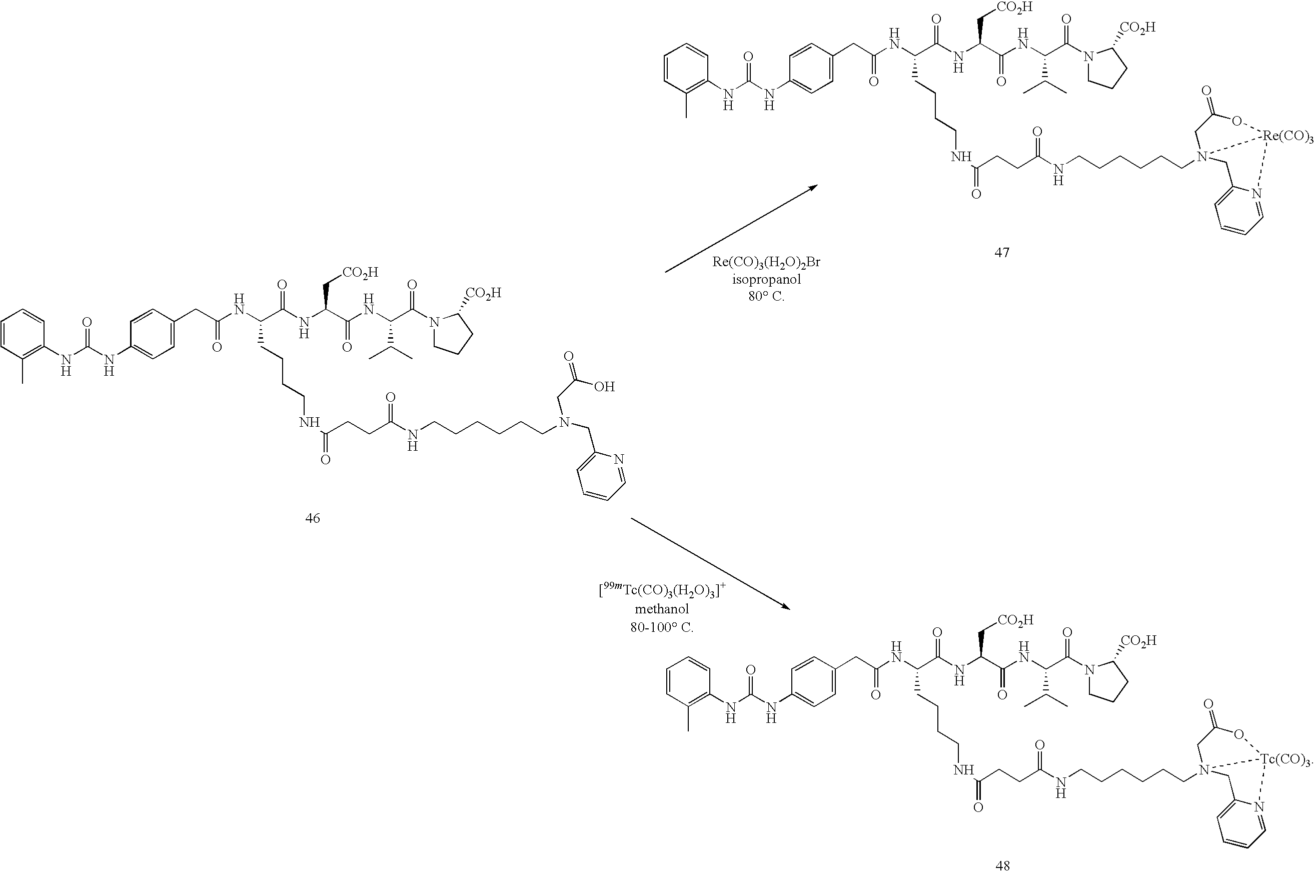

The neutral β-aminoacid PAMA analogs can be likewise prepared by related synthetic routes. Hence, the PAMA ligands (49), (50), and (51) can be prepared and utilized to prepare the rhenium and ^{99m}Tc analogs.

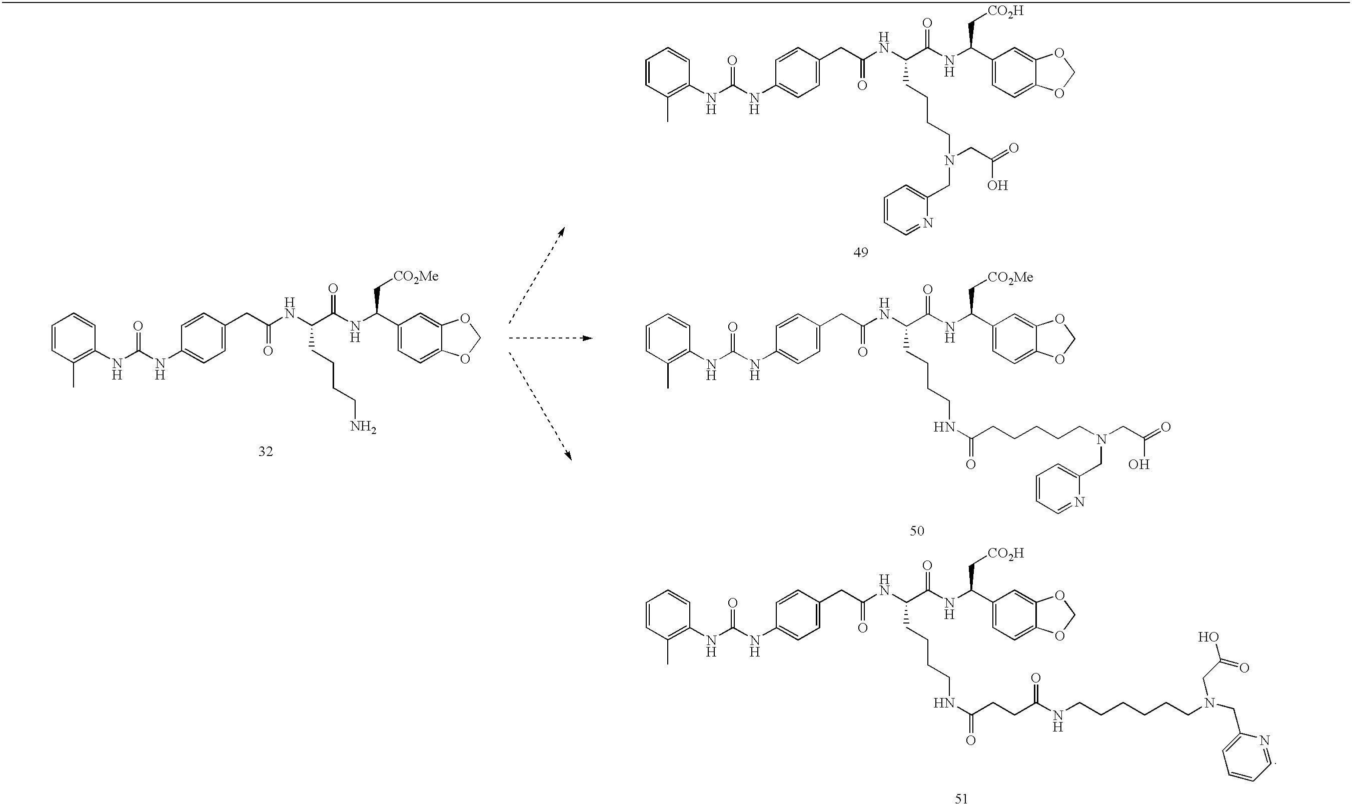

Complexation of (49), (50) and (51) to afford the rhenium complexes (52), (53), and (54) is accomplished as described above and is shown in the scheme below.

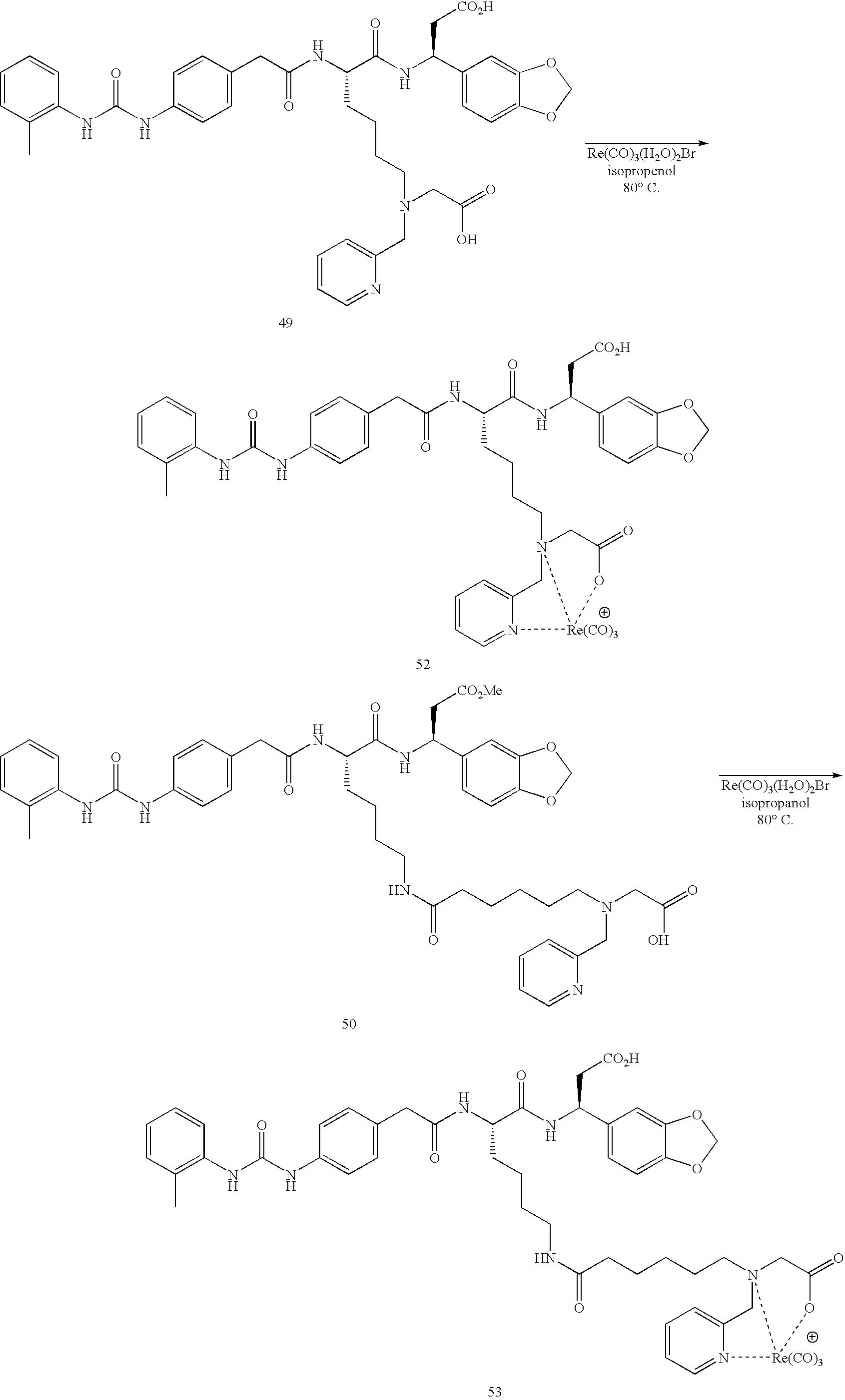

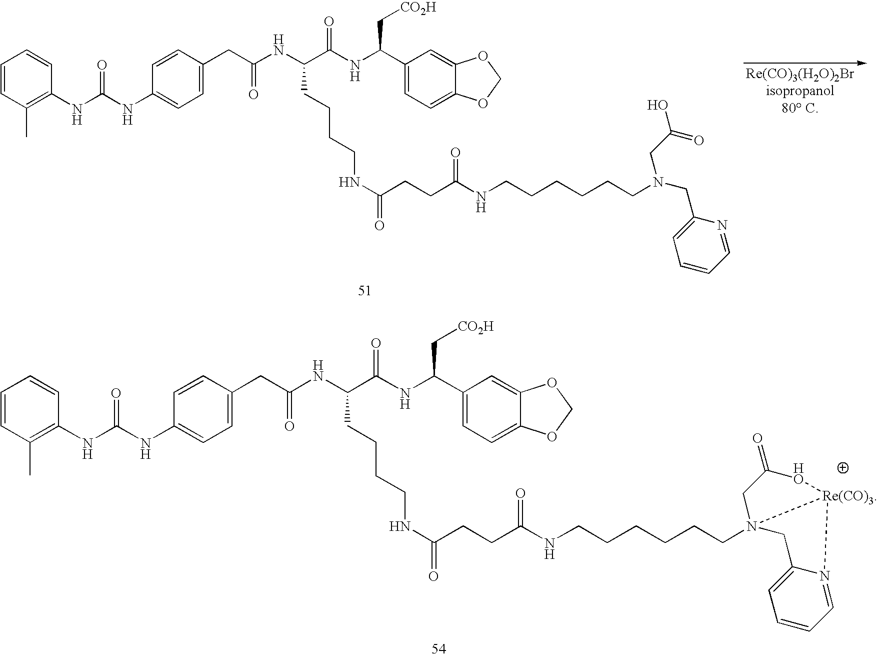

The radioactive neutral PAMA ^{99m}Tc analogs (55), (56) and (57) can be likewise prepared as shown below and described below.

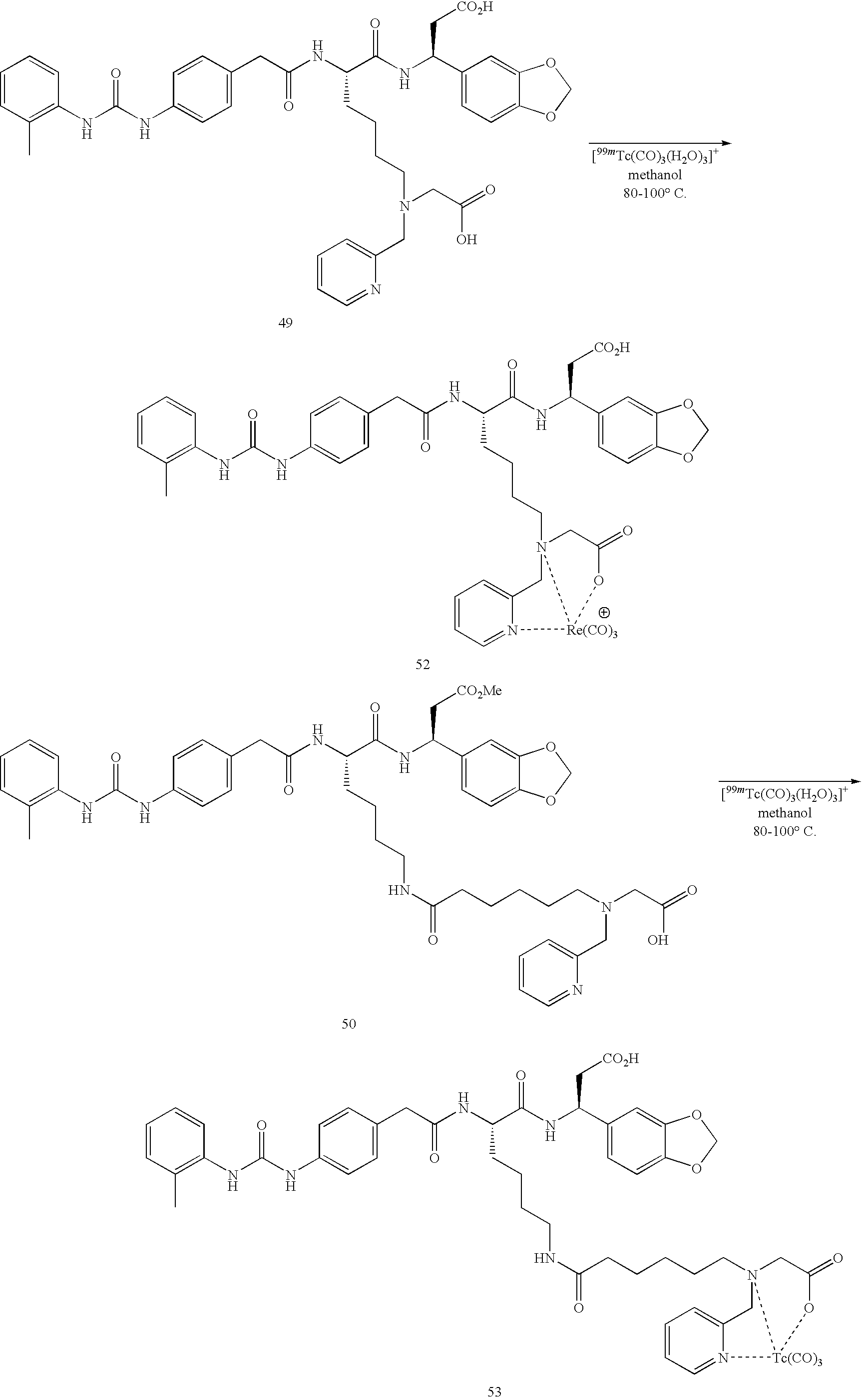

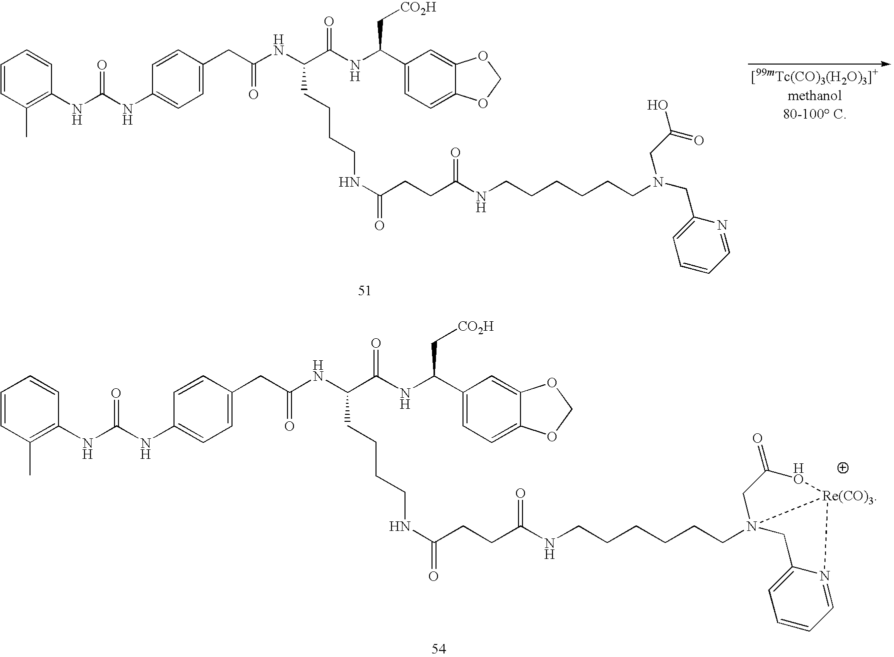

The synthesis of the radioiodine analogs can be accomplished as follows. Coupling of the β-amino acid, (58) to the protected FMOC lysine analog (59) followed by deprotection of the BOC protecting group with TFA affords (60) which is converted to (61) upon coupling with the active ester of the diaryl urea (9). Removal of the FMOC protecting group affords (62).

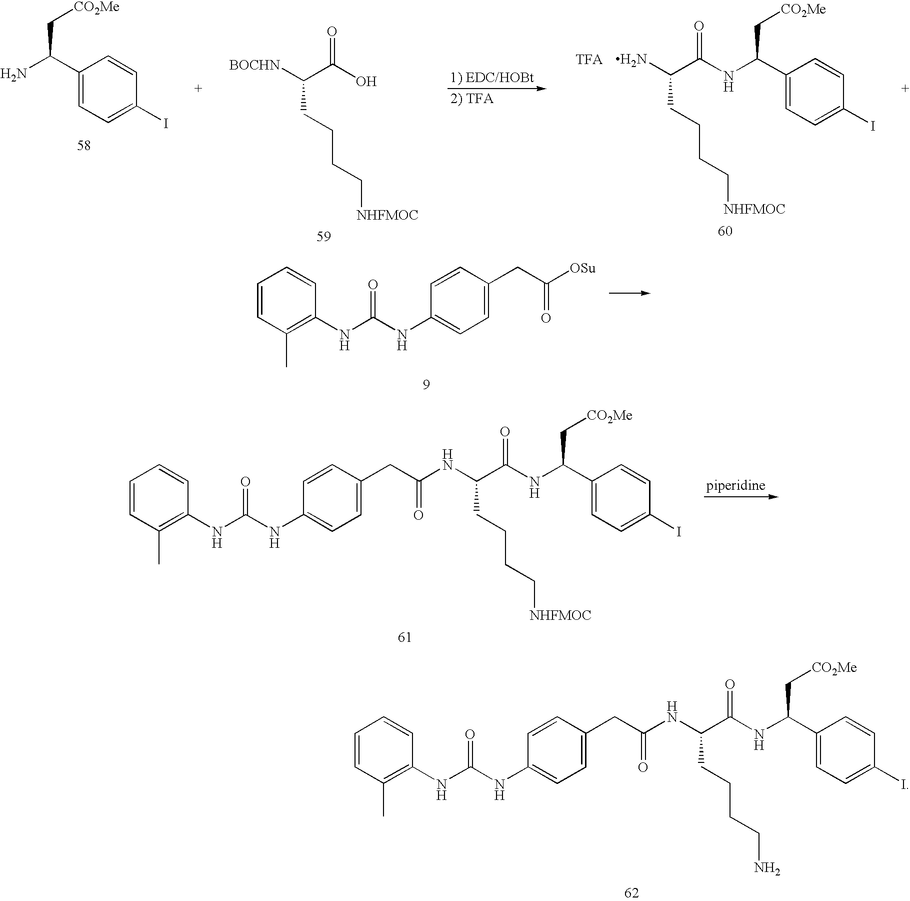

Acylation of the free nitrogen of (62) with simple acyl halide and chloroformates such as methyl chloroformate affords (63). Conversion of (63) to the trimethylstannane intermediate affords (64). Hydrolysis of the methyl ester protecting group affords (65).

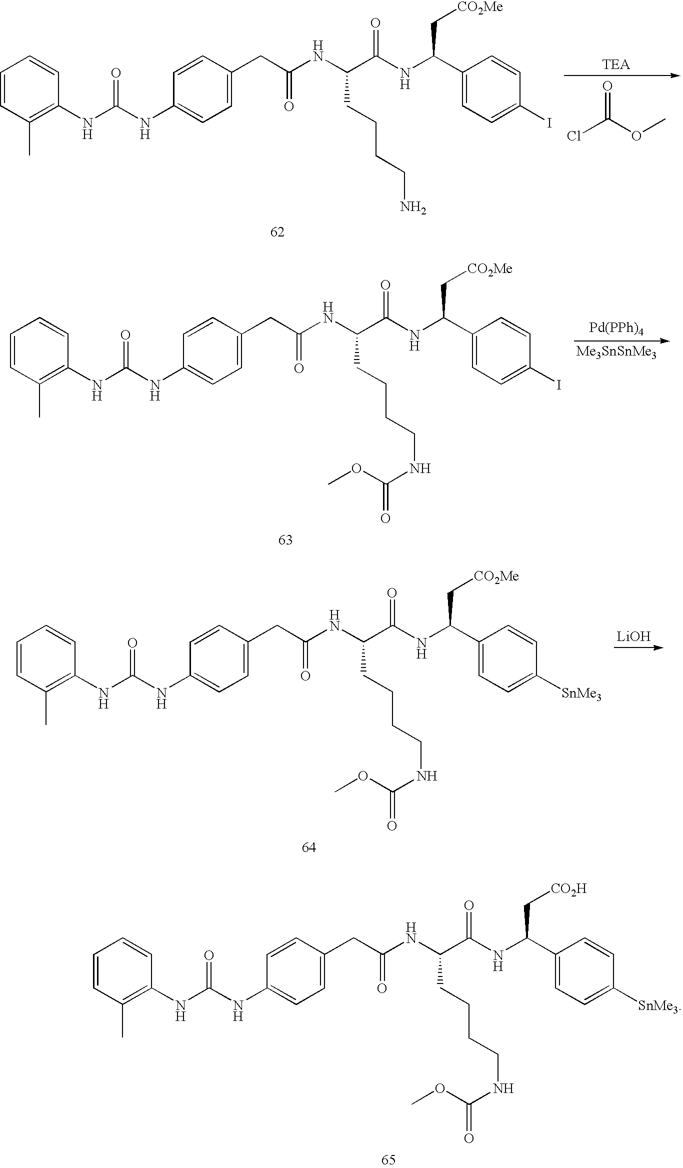

The conversion of (65) to radioiodine species like the ¹²³I analog (66) can be accomplished as shown below to afford the desired radioactive iodine analog.

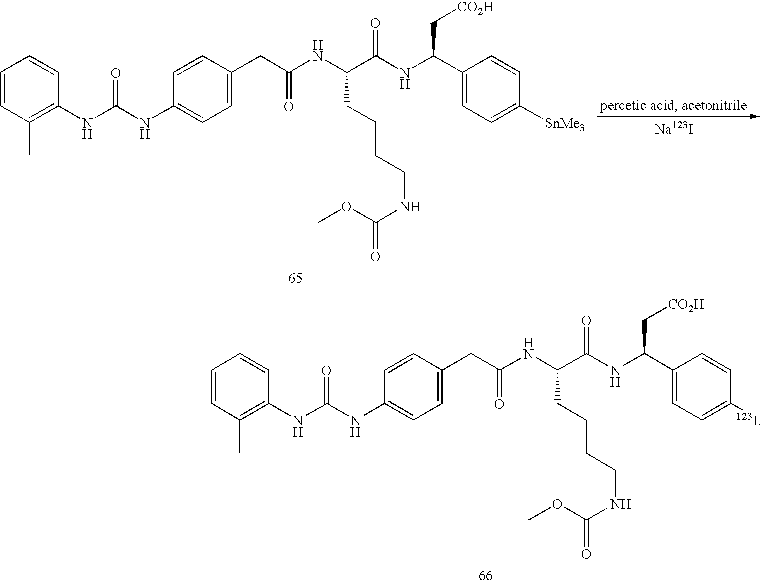

### Radiolabeling Procedure for the Synthesis of 99mTc Analogs:

[^{99m}Tc(CO)₃(H₂O)₃]⁺ was prepared using the Isolink® radiolabeling kits. Sodium Pertechnetate, 7400 MBq (200 mCi), in saline (2.5 mL) was added to an Isolink® radiolabeling kit and the vial was placed in an oil bath at 100° C. The reaction was heated for 45 minutes and 1N HCl (200 μL) was then added to neutralize the reaction mixture. The product, [^{99m}Tc(CO)₃(H₂O)₃]⁺, was removed from the vial via syringe and added to another vial containing the desired ligand to be complexed (200 μL of a 1 mg/mL solution in methanol) followed by an additional amount of methanol (0.3 mL). The reaction was heated for 1 hour at 80° C. and the crude reaction was injected on the HPLC to determine RCY and specific activity.

### In-Vitro Binding Assay:

The compounds can be evaluated in-vitro for their binding affinity to VLA-4 as per the methods described and referenced in J. Med. Chem. 1999, 42, 920, which is incorporated herein by reference.

### Biodistribution Studies in Rodents:

A quantitative analysis of the tissue distribution of ¹²³I-MIP-1072 was performed in separate groups of male NCr Nude^{-/-} mice bearing PSMA positive LNCaP xenografts (approximately 100-200 mm³) administered via the tail vein as a bolus injection (approximately 2 μCi/mouse) in a constant volume of 0.05 ml. The animals (n=5/time point) were euthanized by asphyxiation with carbon dioxide at 0.25, 1, 2, 4, 8, and 24 hours post injection. Tissues (blood, heart, lungs, liver, spleen, kidneys, adrenals, stomach, large and small intestines (with contents), testes, skeletal muscle, bone, brain, adipose, and tumor) were dissected, excised, weighed wet, transferred to plastic tubes and counted in an automated γ-counter (LKB Model 1282, Wallac Oy, Finland). To compare uptake of ¹²³I-MIP-1072 in LNCaP versus PC3 tumors, and to demonstrate that the compound was on mechanism via competition with 2-(phosphonomethyl)-pentanedioic acid (PMPA), some mice bearing either LNCaP or PC3 xenografts were pretreated with 50 mg/kg PMPA 5 minutes prior to injection with ¹²³I-MIP-1072 and selected tissues were harvested at 1 hour post injection.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A complex of formula I, its stereoisomer or pharmaceutically acceptable salt: 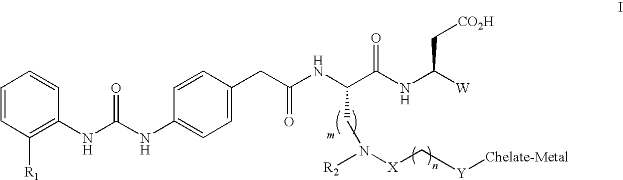
wherein:
W is a group that is A, B, or C: 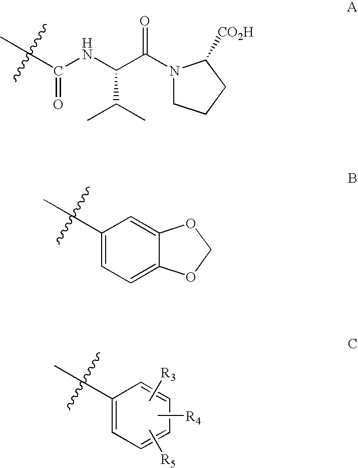
R₁ is hydrogen, alkyl or alkoxy;
R₂ is a bond, hydrogen or alkyl;
R₃, R₄ and R₅ are independently hydrogen, iodine, alkyl, alkoxy, hydroxyl, amino, aminoalkyl, dialkylamino, or carboxyl;
X is a bond, C═O, O═C—O, or CH₂;
Y is a bond, CH₂, or O;
m is an integer ranging from 1 to 6;
n is an integer ranging from 0 to 6;
Metal represents a metallic moiety comprising a radionuclide; and
Chelate represents a chelating moiety that coordinates with said radionuclide to form the complex.

2. The complex of claim 1, wherein said radionuclide is technetium, rhenium, yttrium, indium, gallium, gadolinium, or copper.

3. The complex of claim 1, wherein said Chelate is 1,4,7,10-tetraazacyclododecane-1,4,7,10-tetraacetic acid (DOTA), diethylenetriaminepentaacetic acid (DTPA), pyridylmethylene amine (PDA), quinolinemethylene amine, isoquinoline amine, pyridine-2-ylmethylamino acetic acid (PAMA), isoquinolin-3-ylmethylamino acetic acid, thiazol-2-ylmethyl amine, thiazol-2-ylmethylamino acetic acid, N-methylimidazole(methylene)amine, N-methylimidazole(methylene)amino acetic acid NOTA, Hynic, MAG3, N₂S₂, MAMA, or DADT.

4. The complex of claim 1, wherein said Metal-Chelate moiety is D, E, F, G, H, or J: 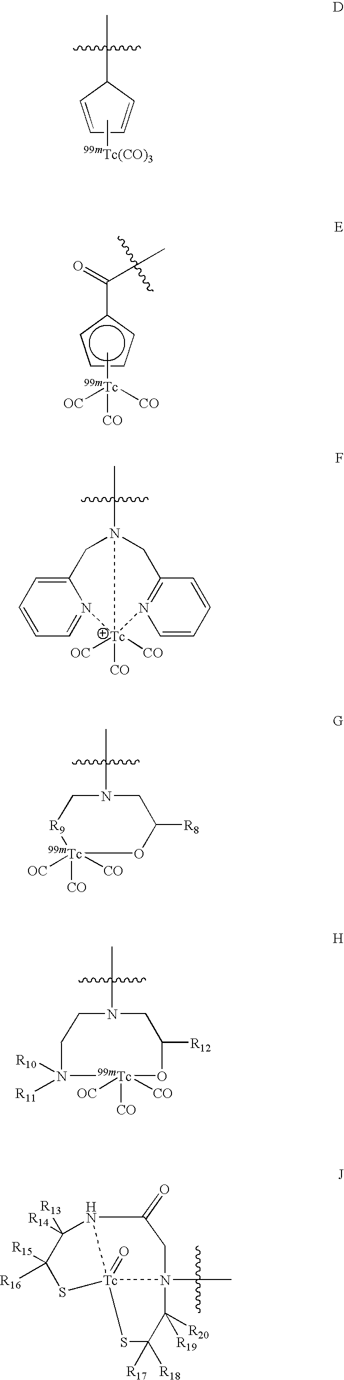
wherein:
R₈ is selected from the group consisting of O, H, OH, alkoxy, or O-alkyl;
R₉ is a pharmaceutically acceptable 5 or 6-member heterocyclic ring;
R₁₀ and R₁₁ are each independently hydrogen, alkyl, or substituted alkyl;
R₁₂ is selected from the group consisting of aryl, alkyl, or heterocycle; and
R₁₃, R₁₄, R₁₅, R₁₆, R₁₇, R₁₈, R₁₉, R₂₀ are independently hydrogen or methyl.

5. The complex of claim 1 which has the structure of I-a:

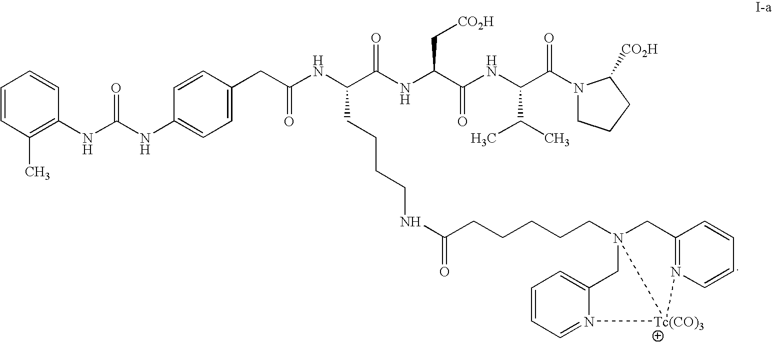

6. The complex of claim 1 which has the structure of I-b:

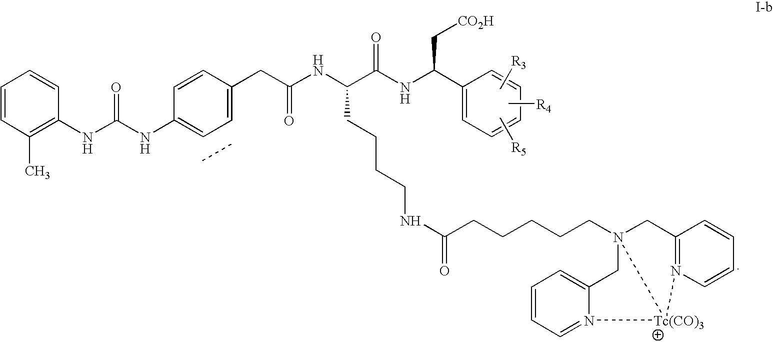

7. The complex of claim 1 which has the structure of I-c:

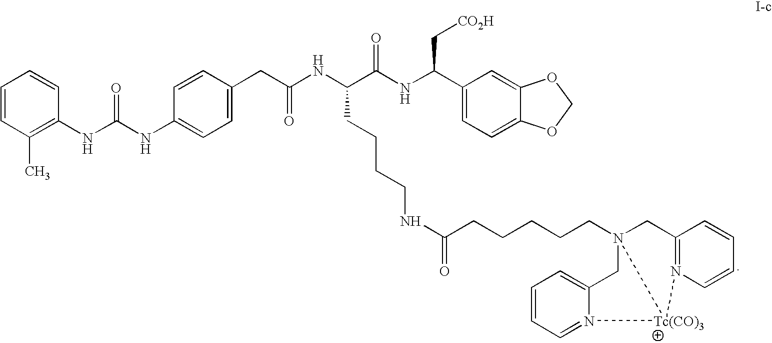

8. The complex of claim 1 which has the structure of I-d:

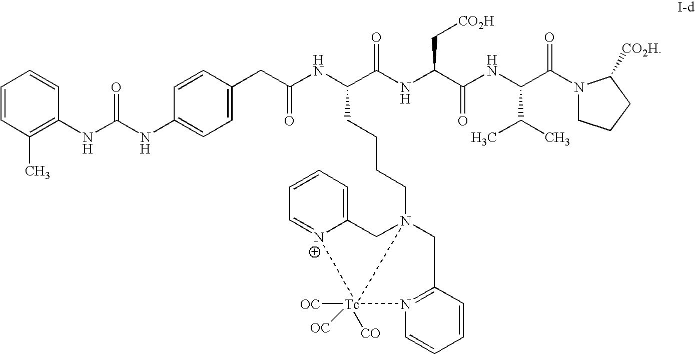

9. The complex of claim 1 which has the structure of formula I-e:

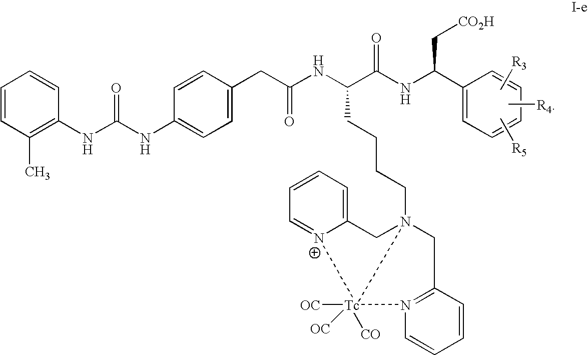

10. The complex of claim 1 which has the structure of formula I-f:

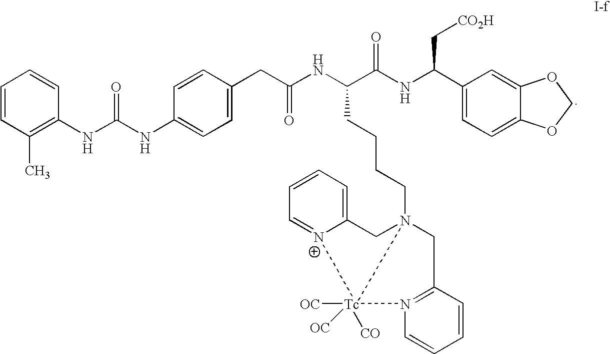

11. A method of imaging tissue of a mammal which expresses VLA-4 comprising administering to said mammal an effective amount of a complex of formula I, its stereoisomer or pharmaceutical salt: 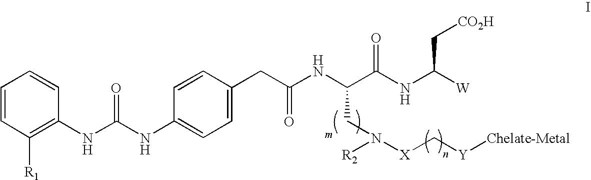
wherein:
W is a group that is A, B, or C: 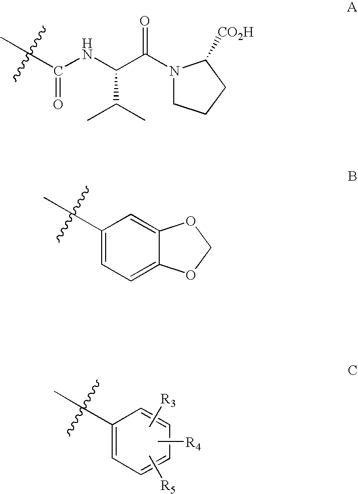
R₁ is hydrogen, alkyl or alkoxy;
R₂ is a bond, hydrogen or alkyl;
R₃, R₄ and R₅ are independently hydrogen, iodine, alkyl, alkoxy, hydroxyl, amino, aminoalkyl, dialkylamino, or carboxyl;
X is a bond, C═O, O═C—O, or CH₂;
Y is a bond, CH₂, or O;
m is an integer ranging from 1 to 6;
n is an integer ranging from 0 to 6;
Metal represents a metallic moiety comprising a radionuclide; and
Chelate represents a chelating moiety that coordinates with said radionuclide to form the complex.

12. The method of claim 11, wherein said radionuclide is technetium, rhenium, yttrium, indium, gallium, gadolinium, or copper.

13. The method of claim 11, wherein said Chelate is 1,4,7,10-tetraazacyclododecane-1,4,7,10-tetraacetic acid (DOTA), diethylenetriaminepentaacetic acid (DTPA), pyridylmethylene amine (PDA), quinolinemethylene amine, isoquinoline amine, pyridine-2-ylmethylamino acetic acid (PAMA), isoquinolin-3-ylmethylamino acetic acid, thiazol-2-ylmethyl amine, thiazol-2-ylmethylamino acetic acid, N-methylimidazole(methylene)amine, N-methylimidazole(methylene)amino acetic acid NOTA, Hynic, MAG3, N₂S₂, MAMA, or DADT.

14. The method of claim 11, wherein said Metal-Chelate moiety is D, E, F, G, H, or J: 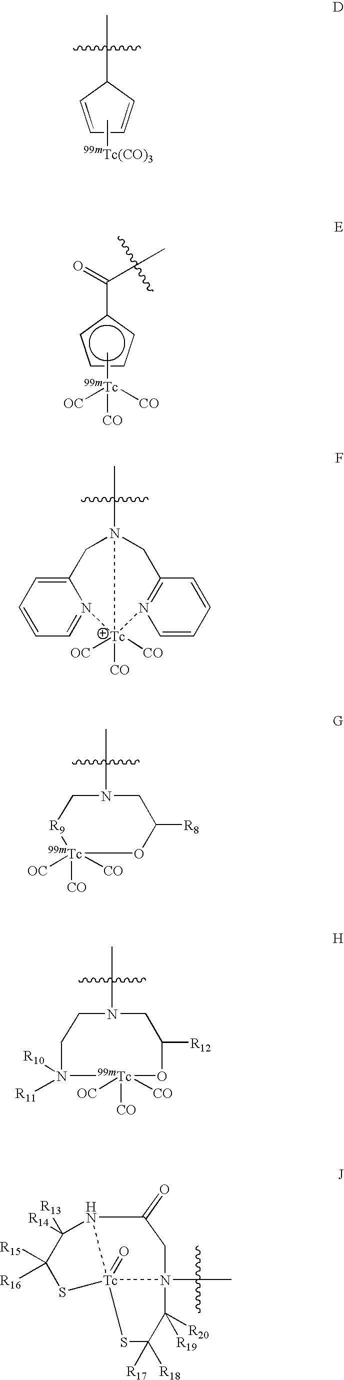
wherein:
R₈ is selected from the group consisting of O, H, OH, alkoxy, or O-alkyl;
R₉ is a pharmaceutically acceptable 5 or 6-member heterocyclic ring;
R₁₀ and R₁₁ are each independently hydrogen, alkyl, or substituted alkyl;
R₁₂ is selected from the group consisting of aryl, alkyl, or heterocycle; and
R₁₃, R₁₄, R₁₅, R₁₆, R₁₇, R₁₈, R₁₉, R₂₀ are independently hydrogen or methyl.

15. The method of claim 11 in which said complex is:

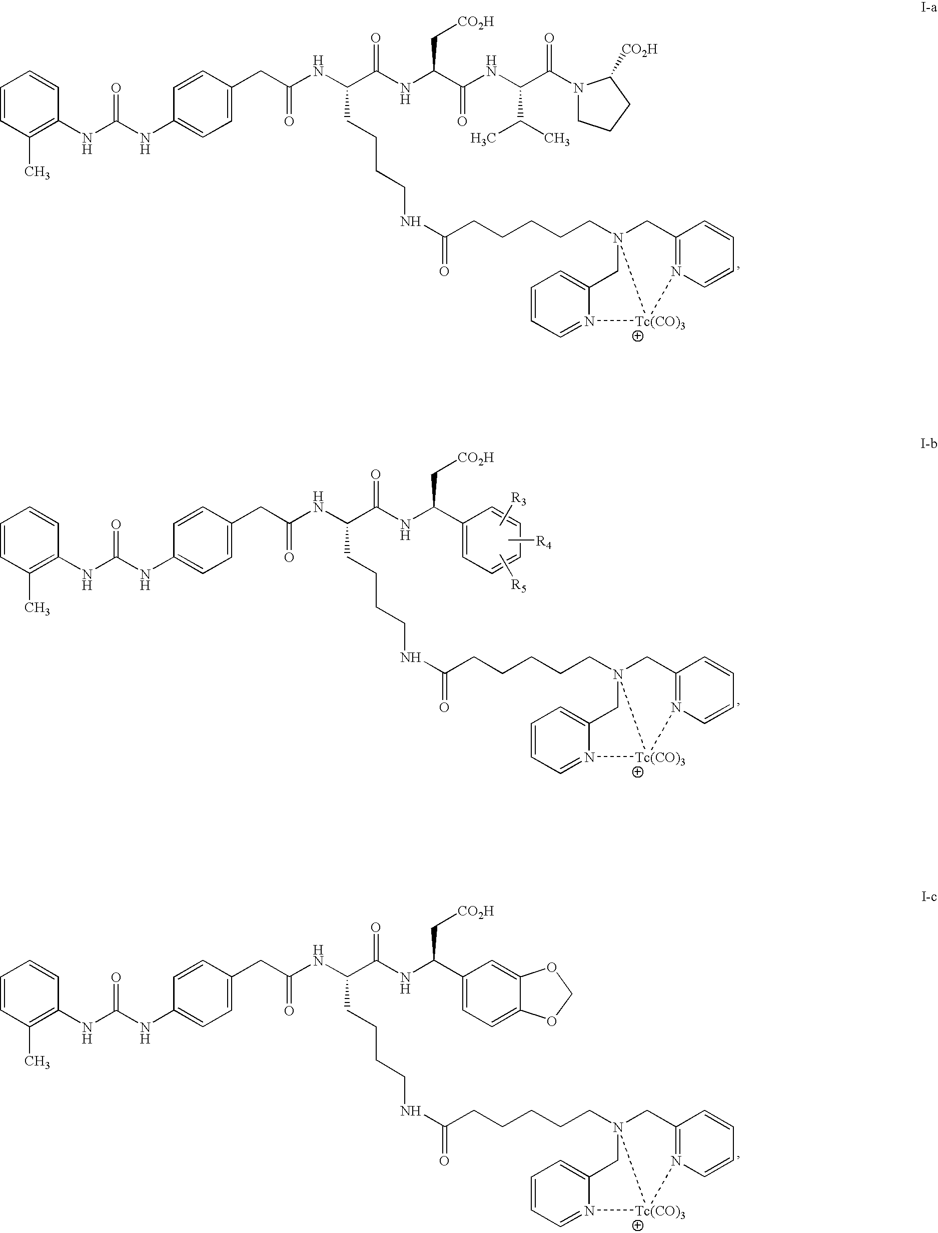

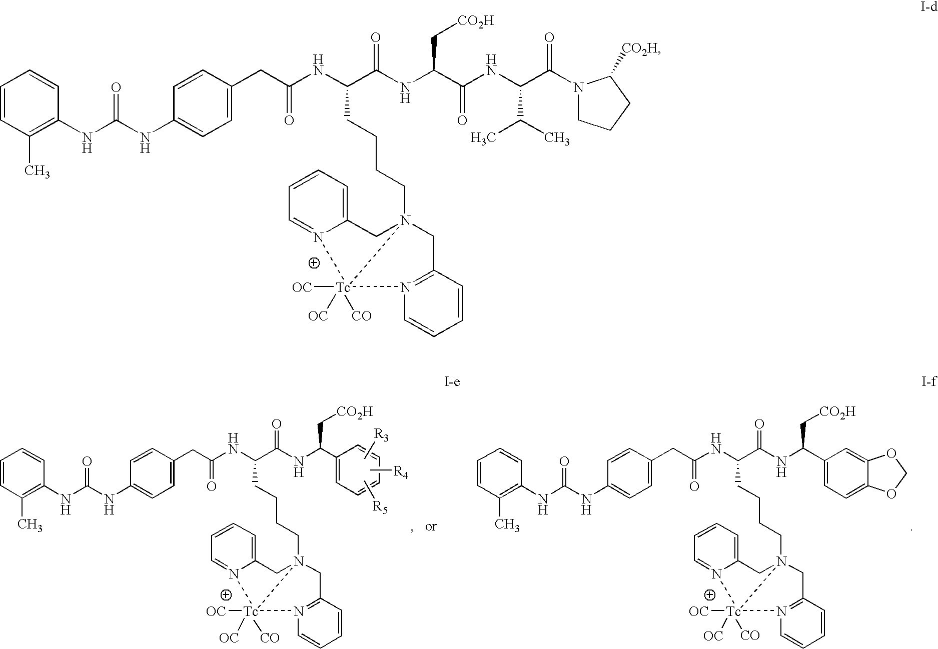

16. A method of treating a mammal suffering a disease which is characterized by overexpression of VLA-4, the method comprising administering to said mammal a therapeutically effective amount of a complex of formula I, its stereoisomer or pharmaceutical salt: 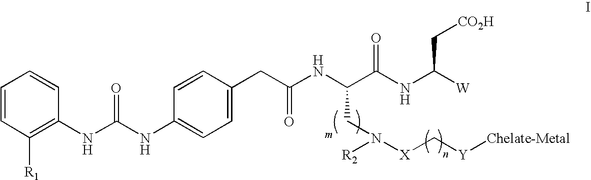
wherein:
W is a group that is A, B, or C: 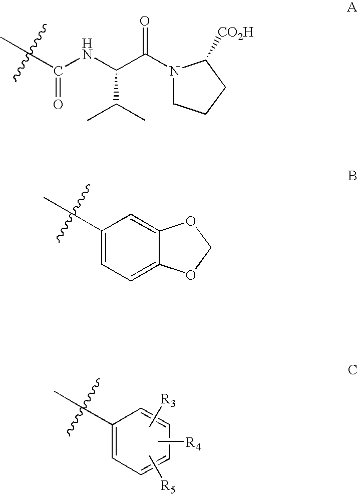
R₁ is hydrogen, alkyl or alkoxy;
R₂ is a bond, hydrogen or alkyl;
R₃, R₄ and R₅ are independently hydrogen, iodine, alkyl, alkoxy, hydroxyl, amino, aminoalkyl, dialkylamino, or carboxyl;
X is a bond, C═O, O═C—O, or CH₂;
Y is a bond, CH₂, or O;
m is an integer ranging from 1 to 6;
n is an integer ranging from 0 to 6;
Metal represents a metallic moiety comprising a radionuclide; and
Chelate represents a chelating moiety that coordinates with said radionuclide to form the complex.

17. The method of claim 16, wherein said radionuclide is technetium, rhenium, yttrium, indium, gallium, gadolinium, or copper.

18. The method of claim 16, wherein said Chelate is 1,4,7,10-tetraazacyclododecane-1,4,7,10-tetraacetic acid (DOTA), diethylenetriaminepentaacetic acid (DTPA), pyridylmethylene amine (PDA), quinolinemethylene amine, isoquinoline amine, pyridine-2-ylmethylamino acetic acid (PAMA), isoquinolin-3-ylmethylamino acetic acid, thiazol-2-ylmethyl amine, thiazol-2-ylmethylamino acetic acid, N-methylimidazole(methylene)amine, N-methylimidazole(methylene)amino acetic acid NOTA, Hynic, MAG3, N₂S₂, MAMA, or DADT.

19. The method of claim 16 in which said Metal-Chelate moiety is D, E, F, G, H, or J: 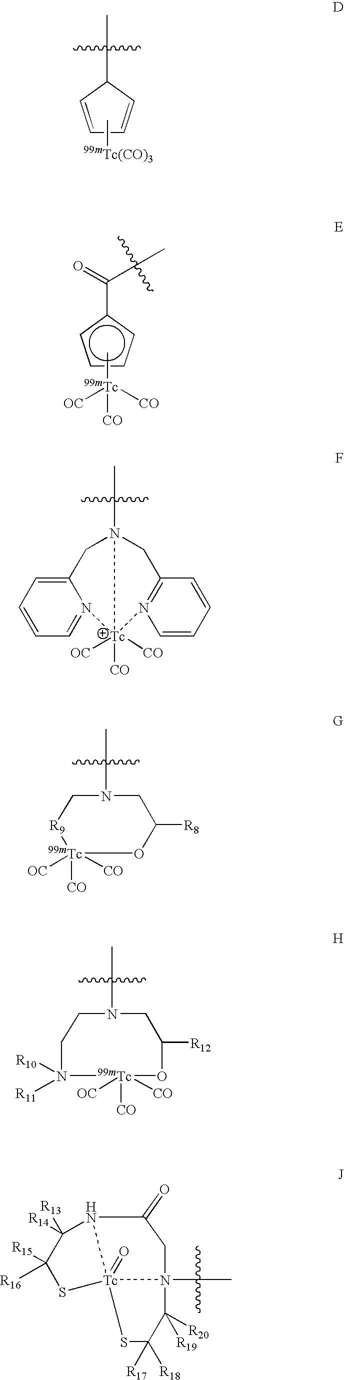
wherein:
R₈ is selected from the group consisting of O, H, OH, alkoxy, or O-alkyl;
R₉ is a pharmaceutically acceptable 5 or 6-member heterocyclic ring;
R₁₀ and R₁₁ are each independently hydrogen, alkyl, or substituted alkyl;
R₁₂ is selected from the group consisting of aryl, alkyl, or heterocycle; and
R₁₃, R₁₄, R₁₅, R₁₆, R₁₇, R₁₈, R₁₉, R₂₀ are independently hydrogen or methyl.

20. The method of claim 16 in which said complex is:

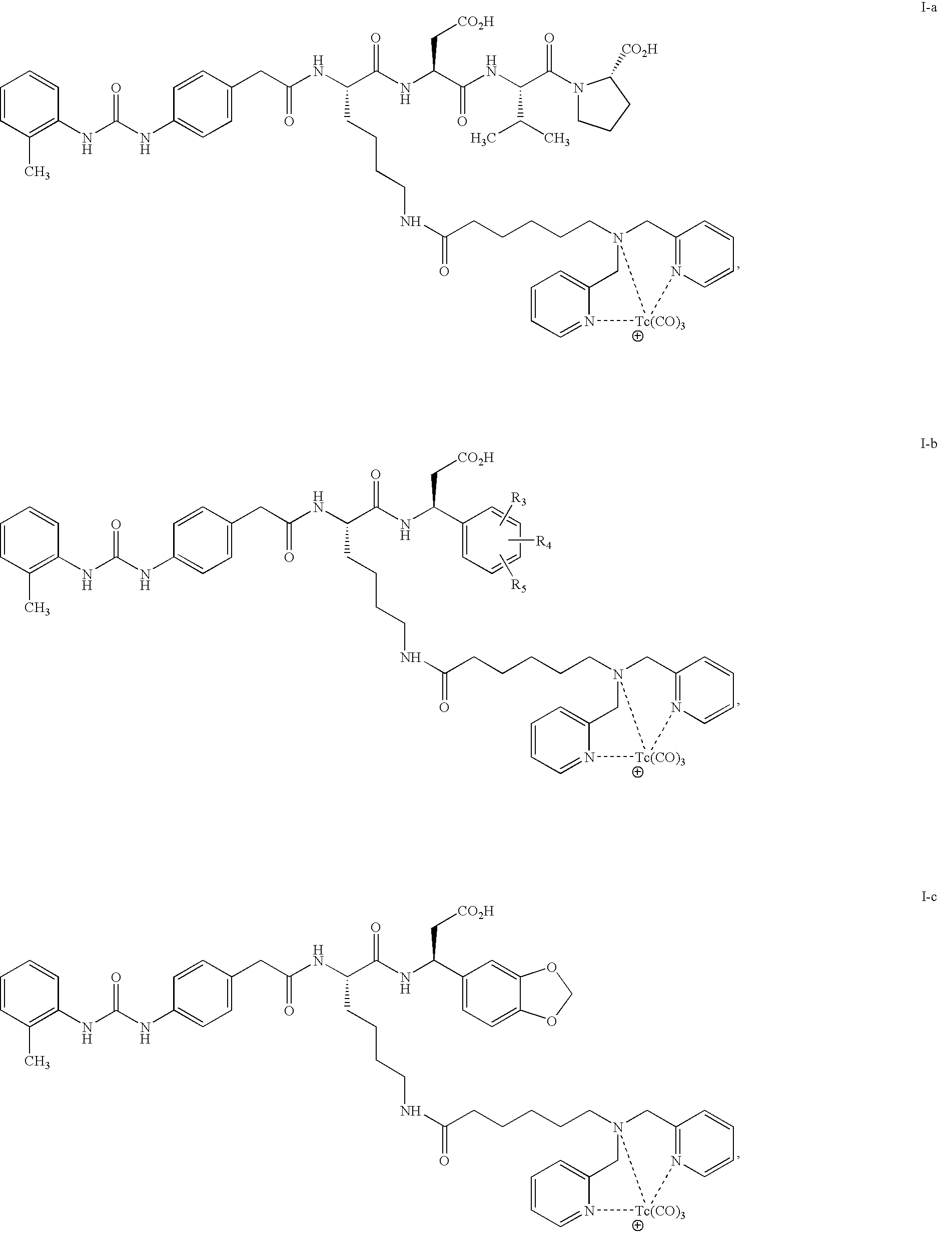

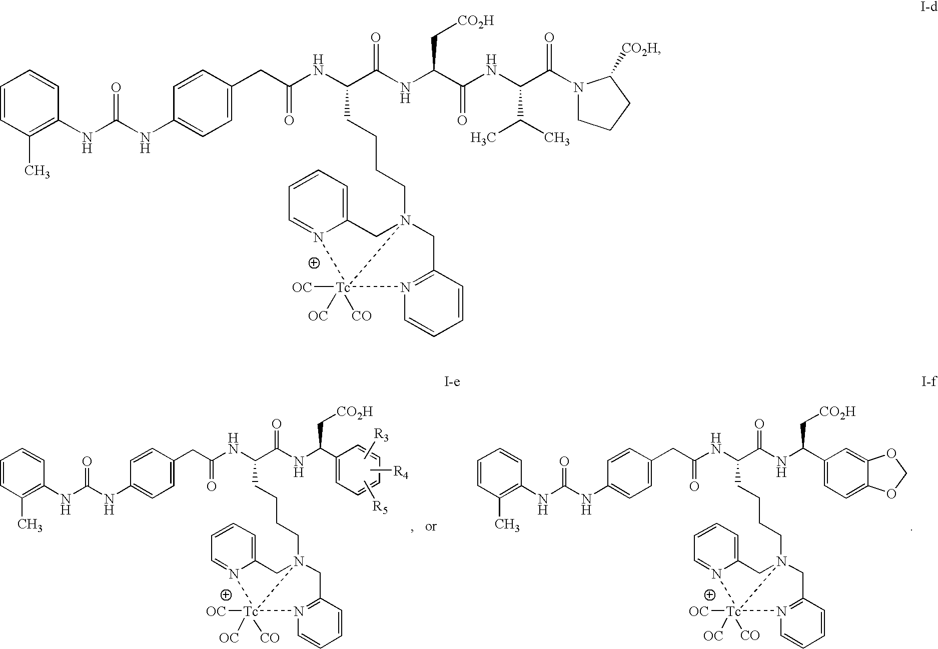

21. A kit comprising a complex of formula I, its stereoisomer or pharmaceutical salt: 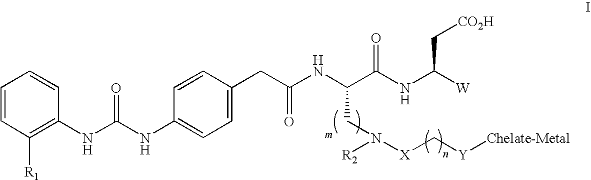
wherein:
W is a group that is A, B, or C: 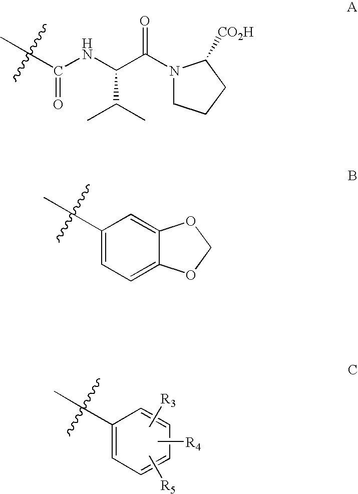
R₁ is hydrogen, alkyl or alkoxy;
R₂ is a bond, hydrogen or alkyl;
R₃, R₄ and R₅ are independently hydrogen, iodine, alkyl, alkoxy, hydroxyl, amino, aminoalkyl, dialkylamino, or carboxyl;
X is a bond, C═O, O═C—O, or CH₂;
Y is a bond, CH₂, or O;
m is an integer ranging from 1 to 6;
n is an integer ranging from 0 to 6;
Metal represents a metallic moiety comprising a radionuclide;
Chelate represents a chelating moiety that coordinates with said radionuclide to form the complex; and
a pharmaceutically acceptable carrier.
